SEQUENCE LISTING (50 sequences)

SEQ ID NO: 1 - DNA, 101 bp, Allium cepa
catggcaagaaaaatgttccaagcttcttgaaacatctccagacaattggtctttcaaaagtgattccgaaacctctacttcgaagtacattgcttcttta

SEQ ID NO: 2 - DNA, 101 bp, Allium cepa
catggcaagaaaaatgttccaagcttcttgaaacatctccagacaattgggctttcaaaagtgattccgaaacctctacttcgaagtacattgcttcttta

SEQ ID NO: 3 - DNA, 101 bp, Allium cepa
ttgataaaagcgatattgttggggaagtatcctgcagaatttttgtggccatgaaaaggaagatgagtggaagctaaatgcattggaaattgtgtttggcc

SEQ ID NO: 4 - DNA, 101 bp, Allium cepa
ttgataaaagcgatattgttggggaagtatcctgcagaatttttgtggccgtgaaaaggaagatgagtggaagctaaatgcattggaaattgtgtttggcc

SEQ ID NO: 5 - DNA, 101 bp, Allium cepa
gacggataattcagaacagggaggaagtaagcagcatgtgataatcaacacagaggatagagcttctgaggtgggtaaagctgatgcgaatcgtcccacgc

SEQ ID NO: 6 - DNA, 101 bp, Allium cepa
gacggataattcagaacagggaggaagtaagcagcatgtgataatcaacatagaggatagagcttctgaggtgggtaaagctgatgcgaatcgtcccacgc

SEQ ID NO: 7 - DNA, 101 bp, Allium cepa
tgatgctatctggaagattacaaatttatagatggtatgggatgttggaatagcagattggtgctgaatttagtactaccaaaggacagatgacgccgctt

SEQ ID NO: 8 - DNA, 101 bp, Allium cepa
tgatgctatctggaagattacaaatttatagatggtatgggatgttggaacagcagattggtgctgaatttagtactaccaaaggacagatgacgccgctt

SEQ ID NO: 9 - DNA, 101 bp, Allium cepa
ctcaggaatgagatttgcatcagcatcctcaggatcaatttcttgatcatcagattgcattccataattaaacagcagctcgtgccacttcatgtcatcaa

SEQ ID NO: 10 - DNA, 101 bp, Allium cepa
ctcaggaatgagatttgcatcagcatcctcaggatcaatttcttgatcattagattgcattccataattaaacagcagctcgtgccacttcatgtcatcaa

SEQ ID NO: 11 - DNA, 101 bp, Allium cepa
cctgagcgatgtaaaaggaagagatagagtatgggagttgagaaactgtaaccatgtttttcataaaggatgcctggacaaatggttagagcatgatgagc

SEQ ID NO: 12 - DNA, 101 bp, Allium cepa
cctgagcgatgtaaaaggaagagatagagtatgggagttgagaaactgtagccatgtttttcataaaggatgcctggacaaatggttagagcatgatgagc

SEQ ID NO: 13 - DNA, 101 bp, Allium cepa
gattgtttagacattcgttgtatttcgcgagatctgctcacgggatagctcattttttaagatttttcgagaaattctacctggatttttgttagggtttt

SEQ ID NO: 14 - DNA, 101 bp, Allium cepa
gattgtttagacattcgttgtatttcgcgagatctgctcacgggatagctgattttttaagatttttcgagaaattctacctggatttttgttagggtttt

SEQ ID NO: 15 - DNA, 101 bp, Allium cepa
aattacactttagcaatcaagagatgattctcaggagaaatatccgaggatgtatacttcaccatttgtgcatccaacccctgatcccttagccacgacat

SEQ ID NO: 16 - DNA, 101 bp, Allium cepa
aattacactttagcaatcaagagatgattctcaggagaaatatccgaggaggtatacttcaccatttgtgcatccaacccctgatcccttagccacgacat

SEQ ID NO: 17 - DNA, 101 bp, Allium cepa
gatgatggtggagcgagaagagaatggttctggtttgtggtttgattggatgggtttgtcgatcgaggtccatgaccattgcttgtggatgcggttccatc

SEQ ID NO: 18 - DNA, 101 bp, Allium cepa
gatgatggtggagcgagaagagaatggttctggtttgtggtttgattggacgggtttgtcgatcgaggtccatgaccattgcttgtggatgcggttccatc

SEQ ID NO: 19 - DNA, 101 bp, Allium cepa
gagcaagatcagttaagattcttaaagctctttgaagacaccgatgagttggatgatgagttggaacaattataagttcaatctactacgccatactttac

SEQ ID NO: 20 - DNA, 101 bp, Allium cepa
gagcaagatcagttaagattcttaaagctctttgaagacaccgatgagttcgatgatgagttggaacaattataagttcaatctactacgccatactttac

SEQ ID NO: 21 - DNA, 101 bp, Allium cepa
acttatttgtaccagatgctagttcatcatacgatctcgatcaacagctcaaaactgtccccacttcatctgacggcaatatcatggtttcttggaatcct

SEQ ID NO: 22 - DNA, 101 bp, Allium cepa
acttatttgtaccagatgctagttcatcatacgatctcgatcaacagctcgaaactgtccccacttcatctgacggcaatatcatggtttcttggaatcct

SEQ ID NO: 23 - DNA, 101 bp, Allium cepa
actcttttcaaaatggcaattccaaaacttgaactttaacttttgttacacgcttaatcacgtcgataatcatgcttagcaccactgccactttctaaatc

SEQ ID NO: 24 - DNA, 101 bp, Allium cepa
actcttttcaaaatggcaattccaaaacttgaactttaacttttgttacatgcttaatcacgtcgataatcatgcttagcaccactgccactttctaaatc

SEQ ID NO: 25 - DNA, 101 bp, Allium cepa
cctgaacattatgcaaaatgtttagccacactttcacgctcatcttcacttggaattcgattgtgttttcgtcagtacagtaaaaataaattcaagctttt

SEQ ID NO: 26 - DNA, 101 bp, Allium cepa
cctgaacattatgcaaaatgtttagccacactttcacgctcatcttcactaggaattcgattgtgttttcgtcagtacagtaaaaataaattcaagctttt

SEQ ID NO: 27 - DNA, 101 bp, Allium cepa
cgtgaaagtgtggctaaacattttgcataatgttcaggagttaaaaccacagcaatgtcatctatacattctgcgctgatcacttgtgagaagggcgtgaa

SEQ ID NO: 28 - DNA, 101 bp, Allium cepa
cgtgaaagtgtggctaaacattttgcataatgttcaggagttaaaaccacggcaatgtcatctatacattctgcgctgatcacttgtgagaagggcgtgaa

SEQ ID NO: 29 - DNA, 101 bp, Allium cepa
taagcttcatgaagctatatatatcacagtaaaacaggttatttttatgggcatggattactttttaaatttgtaagttggttttgtctcccttttggtta

SEQ ID NO: 30 - DNA, 101 bp, Allium cepa
taagcttcatgaagctatatatatcacagtaaaacaggttatttttatggacatggattactttttaaatttgtaagttggttttgtctcccttttggtta

SEQ ID NO: 31 - DNA, 101 bp, Allium cepa
acgtcggcgggagctttctcggtttgatacacgcctaaatagccggttggatcgactctcgcgtagatcggaccgttgccttggattatttgagttttggc

SEQ ID NO: 32 - DNA, 101 bp, Allium cepa
acgtcggcgggagctttctcggtttgatacacgcctaaatagccggttgggtcgactctcgcgtagatcggaccgttgccttggattatttgagttttggc

SEQ ID NO: 33 - DNA, 101 bp, Allium cepa
tatagtgtggcaaaaggtgcattgcatagagcatttgatgagatagtagtagttgaaagaaattgtggtcgagaagagcagagagatccaatcaatattaa

SEQ ID NO: 34 - DNA, 101 bp, Allium cepa
tatagtgtggcaaaaggtgcattgcatagagcatttgatgagatagtagttgttgaaagaaattgtggtcgagaagagcagagagatccaatcaatattaa

SEQ ID NO: 35 - DNA, 101 bp, Allium cepa
tattataattataagcagtgatgtcacattcattaatttgtgcaccctcattattatccctcgatgaaaagtcaattatttcgttaggaatatccgtagac

SEQ ID NO: 36 - DNA, 101 bp, Allium cepa
tattataattataagcagtgatgtcacattcattaatttgtgcaccctcaatattatccctcgatgaaaagtcaattatttcgttaggaatatccgtagac

SEQ ID NO: 37 - DNA, 101 bp, Allium cepa
tgttgcattccccattacccaatatcactgtcacaactatgctccccaactccttgctacactgcaataacatcacatacaacttcacttccccgaacaac

SEQ ID NO: 38 - DNA, 101 bp, Allium cepa
tgttgcattccccattacccaatatcactgtcacaactatgctccccaacaccttgctacactgcaataacatcacatacaacttcacttccccgaacaac

SEQ ID NO: 39 - DNA, 101 bp, Allium cepa
cttcccctcggtaaatattctgttactatcgacaaatgtggagactttgtgactgcacccataaatagtacaatatttggatggcgaatacgtttcattat

SEQ ID NO: 40 - DNA, 101 bp, Allium cepa
cttcccctcggtaaatattctgttactatcgacaaatgtggagactttgtaactgcacccataaatagtacaatatttggatggcgaatacgtttcattat

SEQ ID NO: 41 - DNA, 101 bp, Allium cepa
tactgcccagtcactgcttgtggggatggattcttcgtcttcaagcgatgctcgaattctctcaagatctttctctgaagcttctttgaaattaatgtcct

SEQ ID NO: 42 - DNA, 101 bp, Allium cepa
tactgcccagtcactgcttgtggggatggattcttcgtcttcaagcgatgttcgaattctctcaagatctttctctgaagcttctttgaaattaatgtcct

SEQ ID NO: 43 - DNA, 101 bp, Allium cepa
gatacccaaaaccctgatatgatcgactatctcaaccaagaaaatgattatactgaatcatttatgaaagatactgaaaaattgcagcgaaaattagtgga

SEQ ID NO: 44 - DNA, 101 bp, Allium cepa
gatacccaaaaccctgatatgatcgactatctcaaccaagaaaatgattacactgaatcatttatgaaagatactgaaaaattgcagcgaaaattagtgga

SEQ ID NO: 45 - DNA, 101 bp, Allium cepa
ttatggtcgaaagaagacctattgaactttgtcatagcacctccagtgggtatcttcccacacagcttattatagccgacgtcaaaatacaccagtttgat

SEQ ID NO: 46 - DNA, 101 bp, Allium cepa
ttatggtcgaaagaagacctattgaactttgtcatagcacctccagtgggcatcttcccacacagcttattatagccgacgtcaaaatacaccagtttgat

SEQ ID NO: 47 - DNA, 101 bp, Allium cepa
aaattggctatggagaagatgaagattgatttggcacagaaagataagatcctgtctgcattgctgagaaaatcaaaggctgataatgaagaaaagcatat

SEQ ID NO: 48 - DNA, 101 bp, Allium cepa; identical to SEQ ID NO: 47

SEQ ID NO: 49 - DNA, 101 bp, Allium cepa
cattctgtcatggttatcagtcacatctaatgatgctttaatactttccggattcagaaatgacaaatgtgctccaccaaatgcttctacacatggtttac

SEQ ID NO: 50 - DNA, 101 bp, Allium cepa
cattctgtcatggttatcagtcacatctaatgatgctttaatactttccgaattcagaaatgacaaatgtgctccaccaaatgcttctacacatggtttac

(19) United States
(12) Patent Application Publication
(10) Pub. No.: US 2020/0288658 A1
(43) Pub. Date: Sep. 17, 2020

(54) ONIONS WITH HIGH STORAGE ABILITY, HIGH SOLUBLE SOLIDS CONTENT AND/OR LOW PUNGENCY

(71) Applicant: NUNHEMS B.V., Nunhem (NL)

(72) Inventors: Rick WATSON, Silverton, OR (US); Tyson Ronald HOWELL, Brooks, OR (US); Juan Carlos BREVIS ACUNA, Brooks, OR (US); Ebenezer A. OGUNDIWIN, West Sacramento, CA (US); Cathy M. PHAM-NGUYEN, West Sacramento, CA (US); Elly SOERYAPRANATA, West Sacramento, CA (US)

(21) Appl. No.: 16/852,942

(22) Filed: Apr. 20, 2020

Related U.S. Application Data
(63) Continuation-in-part of application No. 15/920,102, filed on Mar. 13, 2018, now abandoned.
Application No. 15/920,102 is a continuation of application No. 12/864,405, filed on Jul. 23, 2010, now abandoned.
Application No. 12/864,405 is a continuation of application No. 12/020,360, filed on Jan. 25, 2008, now abandoned.
(60) Provisional application No. 61/054,026, filed on May 16, 2008.

(51) Int. Cl. A01H 5/04; A01H 5/06

(52) U.S. Cl. CPC A01H 5/04; A01H 5/06

(57) ABSTRACT

Long-day onion plants, capable of producing onion bulbs comprising ‘high soluble solids’ combined with a ‘sweet taste’ as a result of low pungency, are provided, as are methods for producing such plants, bulbs and seeds. Such onions can be stored for long periods without a loss in quality and without an increase in pungency.

### CROSS-REFERENCE TO RELATED APPLICATIONS

This application is a continuation-in-part of U.S. application Ser. No. 15/920,102, filed Mar. 13, 2018, which is a continuation of U.S. application Ser. No. 12/864,405, filed Jul. 23, 2010 which is a 35 U.S.C. 371 National Phase of PCT Application No. PCT/EP2009/000321, filed Jan. 20, 2009, which claims priority to U.S. application Ser. No. 12/020,360, filed Jan. 25, 2008, and U.S. Provisional Application No. 61/054,026, filed May 16, 2008, the disclosures of each of which are hereby incorporated by reference.

### FIELD OF THE INVENTION

The invention relates to plant breeding and plant improvement, in particular plants of the species Allium cepa (onion) having new quality characteristics and combinations of at least two characteristics selected from ‘high soluble solids content’, ‘low pungency’ (LP) and/or ‘long storage’ (LS), essentially without significant quality loss during storage (e.g., no significant increase in pungency and/or no significant reduction in soluble solid content). Provided are onion bulbs, plants and seeds having these characteristics (both open pollinated and hybrids, especially long-day onions) as well as methods for making these.

### BACKGROUND

The onion plant is believed to originate from West or Central Asia. In Europe it has been known since the bronze ages. The bulbs of the onion plants, —the “onions”—are used in many dishes and have a very healthy reputation. Plant breeding has been focused on yield, appearance, harvestability, storability, flavor and content as onions contain several compounds that have beneficial effects on health. Some of these compounds are most effective when the onion is consumed fresh and their concentrations are often linked with the solids level of onions. A high solids onion that is mild and sweet enough to be consumed without cooking will deliver more health promoting compounds in the diet.

Onion varieties are characterized by day length; “long-day” onion varieties will stop forming tops and begin to form bulbs when the day length reaches 14 to 16 hours while “short-day” onions will start making bulbs in early spring or in autumn/winter when there are only 10 to 12 hours of daylight. “Long-day” onions are usually produced in northern countries or northern states of the USA (north of the 36th parallel) while “short-day” onions are produced in countries or states south of that line. Long-day onion varieties generally have a more pungent flavour than short-day varieties, which are sweet. Long-day varieties also store better and longer than short-day varieties because they have a relatively higher dry matter content or higher percentage of soluble solids (SSC) compared to short-day onions (see e.g., “Onion Planting” publication, obtainable from the Texas A&M University horticulture website at world wide web. http://aggie-horticulture.tamu.edu/plantanswers/publications/onions/oniongro.html). The long storage ability of long-day onion varieties provides the possibility to market onions during late summer, fall and winter (August-March/April) when mild, short-day onions are not available or scarce. Long-day onions are bi-annual for seed production. Seeding for seed production purposes occurs in autumn, possibly, but not necessarily, followed by transplanting in spring. Seed is harvested the next summer. For bulb production long-day onions are seeded early spring, harvested in autumn and subsequently stored over winter. Short-day onions can be seeded in autumn and harvested in spring the next year, or seeded in spring and harvested in early summer of the same year. As the storage ability of short-day onions is low, the availability of these mild onions is restricted to spring-early summer (April-July).

Pungency is the typical onion flavour or taste, caused by the conversion of sulphur containing flavour precursors—alk(en)yl-L-cysteine-sulfoxides (ACSOs)—by the enzyme allinase into thiosulfonates when the onion cells are cut or damaged. A byproduct of this enzymatic process, pyruvate or pyruvatic acid is measured as an indicator of the pungency (Schwimmer and Weston 1961, J. of Agric. Food Chem. 9: 301-4). The amount of pyruvate produced is directly related to onion pungency as determined by taste panels (Schwimmer and Guadagni, 1962, J. Food Sc. 27:94-97).

Pungency is an important commercial trait as consumers favour fresh onions with low pungency and sweet taste. Pungency masks the sweet taste of the sugars, which are present in the onion as part of the water-soluble solids or carbohydrates. Pungency is strongly influenced by the presence or absence of sulphur in the soil or plant nutrients (Randle 1992, Euphytica 59: 151-156 and Randle and Bussard 1993, J. Amer. Soc. Hort. Sci. 118: 766-770), but has also a clear genetic component as shown by Lin (1995, J. Americ. Soc. Hort. Sci. 120: 119-122), Simon (1995, Euphytica 82: 1-8), Wall et al. (1996, Euphytica 87: 133-139) and Wall and Corgan (1999, Euphytica 106: 7-13). Pungency can, therefore, vary between locations and between years.

Dry matter in onions consists of both soluble and insoluble carbohydrates. The soluble solids are in the form of fructose, sucrose, glucose, fructans and other saccharides. The analysis of dry matter can be time consuming and destructive for the bulbs. Several researchers have determined that dry matter content and refractive index (soluble solids content) are positively correlated with the percentage of dry matter and the refractive index determination avoids destruction of the bulbs (Mann and Hoyle, 1945, Proc. Americ. Soc. Hort Sci. 46: 285-292; Foskett and Peterson, 1949, Proc. Americ. Soc. Hort Sci. 55: 314-318). Low pungency in onions is strongly correlated with low dry matter content or a low percentage of soluble solids (see further below). Short-day onions, thus, have a low pungency and a low SSC at harvest, and cannot be stored for long periods. For the fresh onion market in northern countries or northern states of the USA (i.e., for long-day countries), however, there is a long existing need for low pungency varieties. This requires long-day onions that combine the properties ‘low pungency’ with ‘high solids’. Such onions do not yet exist in the art, because there is an alleged genetic linkage between the properties ‘high pungency’ and ‘high (soluble) solids’. Thus, long-day onions have a high pungency and a high SSC, whereby they can be stored throughout the winter.

This linkage between high pungency and high SSC is, for example, illustrated by a study of Galmarini et al. (2001, Mol. Gent. Genomics 265: 543-551) wherein molecular markers which were significant for pungency were also significant for SSC, suggesting that this characteristic may be controlled by the same chromosome region. It implies a genetic linkage or association between these traits, resulting in short-day onions, which generally have a low soluble solid content together with a low pungency and long-day onions having a high soluble solid content combined with high pungency.

Also other studies support the strong linkage between the two traits—SSC and pungency (Schwimmer and Weston, 1961, supra; Randle 1992, supra; Simon 1995 supra; Lin 1995; MacCallum et al. 2001, NZ J. of Crop and Hort. Sci. 29: 149-158; Galmarini 2001, supra). For example Simon (1995, supra) observes a strong correlation between pungency and SSC in the parent lines, the F1, F2 and BC1 generations of a diallel between 4 parent inbred lines. Galmarini et al. (2001, supra) and Havey et al. (2004, Genome 47: 463-468) found a phenotypic and genetic significant positive correlation between solids and pungency in the F3 generation.

Galmarini et al. and Havey et al. suggest that this linkage may be the result of pleiotropic effects. There is physiological evidence for this scenario as the higher accumulation of fructans in high solids onions, because of no hydrolization of fructans to fructose and less water uptake, is associated with greater thiosulfinate concentrations, yielding strong correlations among soluble carbohydrates, pungency and onion-induced in vitro anti platelet activity (OIAA). The increase in water content and free fructose in low solids onions could be responsible for diluting the compounds related to pungency and increase the sweeter and milder taste. The QTL analysis as discussed in these articles shows a strong linkage in one group (E) between dry matter percentage (DM %), pungency and OIAA, while DM % and solids are strongly linked in a different group (D). This implies a strong association between DM %, soluble solids, pungency and OIAA, which would be difficult to overcome.

According to some reports (Shock et al. 2004: “Pungency of Selected Onion Varieties Before and After Storage”, Oregon State University, Malheur Experiment Station Special Report 1055: 45-46) pungency may significantly increase during storage. There is, therefore, a need for onions which have a low pungency and high SSC at harvest and whereby the pungency does not increase significantly during storage.

### SUMMARY OF VARIOUS ASPECTS OF INVENTION

In the current invention, provided herein is an onion plant requiring 14 or more contiguous hours of daylight to initiate bulb formation comprising a bulb having low pungency, particularly such onion plant, wherein said bulb has a PAD measurement at harvest of less than 5.5 μM/g FW pyruvate, less than 5.0 μM/g FW pyruvate, less than 4.5 μM/g FW pyruvate, less than 4.0 μM/g FW pyruvate, less than 3.75 μM/g FW pyruvate, or equal to or less than 3.5 μM/g FW pyruvate.

Also provided herein is any one of the above onion plants, wherein said onion plant is a yellow onion or a Spanish onion. Further provided herein is any one of the above onion plants, wherein said bulb is low pungent at harvest, or wherein said bulb substantially maintains low pungency after storage for about 2 months, such as any of the above onion plants, wherein a PAD measurement after storage is increased less than 10% from a PAD measurement at harvest, wherein said bulb substantially maintains low pungency after storage for about 4 months, or wherein a PAD measurement after storage is increased less than 10% from a PAD measurement at harvest.

Further provided is any one of the above onion plants, wherein said bulb substantially maintains low pungency after storage for about 6 months, such as any one of the above onion plants, wherein a PAD measurement after storage is increased less than 10% from a PAD measurement at harvest. Also provided herein is any one of the above onion plants, wherein said onion plant requires 14 or more contiguous hours of light for 2 or more, 4 or more, or 7 or more days to initiate bulb formation.

In accordance with this invention, provided herein is a part of an onion plant requiring 14 or more contiguous hours of light to initiate bulb formation, wherein said plant comprises a bulb having a PAD measurement of less than 5.5 μM/g FW pyruvate, preferably less than or equal to 3.5 μM/g FW pyruvate, such as such plant part, which is selected from the group consisting of a seed, bulb, leaf, pollen, or an ovule.

Further provided herein is a cell, a protoplast, or a tissue culture of cells derived or obtained from any one of the above onion plants, such as a tissue culture from a tissue selected from the group consisting of leaf, pollen, embryo, bulb, anther, flower, bud, and meristem.

Also provided herein is a long-day onion plant comprising a bulb having low pungency, such as a Spanish-type onion plant comprising a bulb having low pungency.

Further provided herein is an onion bulb from a onion plant requiring 14 or more contiguous hours of light to initiate bulb formation comprising a PAD measurement less than about 5.5 μM/g FW pyruvate, less than about 5.0 μM/g FW pyruvate, less than about 4.5 μM/g FW pyruvate, less than about 4.0 μM/g FW pyruvate, less than about 3.75 μM/g FW pyruvate, or equal to or less than 3.5 μM/g FW pyruvate, such as any one of said bulbs which is a yellow onion bulb, or a Spanish onion bulb.

Also provided herein is a container of onion bulbs from onion plants requiring 14 or more contiguous hours of light to initiate bulb formation comprising an average PAD measurement of less than about 5.5 μM/g FW pyruvate, less than about 5.0 μM/g FW pyruvate, less than about 4.5 μM/g FW pyruvate, or less than or equal to 3.5 μM/g FW pyruvate. Further provided is any such container, wherein at least 75%, at least 85%, or at least 95% of said onion bulbs have a PAD measurement of less than about 5.5 μM/g FW pyruvate. Included herein is any one of the above containers, wherein said container is selected from a bag, a can, a box, and a flat, or a container that contains 1 pound or 5 pounds of onion bulbs. Further included herein is any one of the above containers, wherein said container is in a store, such as a grocery store.

In accordance with the current invention is also provided a seed of an onion plant requiring 14 or more contiguous hours of light to initiate bulb formation, wherein said seed is capable of producing an onion plant having a bulb comprising a PAD measurement of less than about 5.5 μM/g FW pyruvate, less than about 5.0 μM/g FW pyruvate, less than about 4.5 μM/g FW pyruvate, less than about 4.0 μM/g FW pyruvate, or equal to or less than 3.5 μM/g FW pyruvate.

Also provided herein is a container of seeds of an onion plant requiring 14 or more contiguous hours of light to initiate bulb formation wherein onion bulbs from greater than 50% of said seeds are low pungency onions, wherein a population of onion bulbs from said seeds contain an average PAD measurement of less than about 5.5 μM/g FW pyruvate, or less than about 5.0 μM/g FW pyruvate, such as such container which comprises at least 100 or 1000 seeds. Such container can be a bag, a box, or a packet. Further provided herein is a any one of the above container of seeds, wherein bulbs from greater than 75%, greater than 85% or greater than 95%, of said seeds are low pungency onions.

Also provided herein is a method of producing a hybrid onion seed comprising: crossing a low pungency onion plant requiring 14 or more hours of light to initiate bulb formation with another onion plant; and obtaining F1 onion seed. Further provided herein is such method, wherein said low pungency onion is an onion line designated I37853B, I37554A, or I37554B, deposited under Accession Nos. PTA-9053, PTA-9054 and PTA-9055, respectively.

In accordance with the present invention, provided herein is a seed of I37853B, a sample of said seed having been deposited under Accession No. PTA-9053, an onion plant grown from said seed, an onion plant part from such onion plant, such as pollen, protoplast, an ovule, or a cell. Also provided herein is a tissue culture of cells obtained from said plant, such as a tissue culture of cells from a tissue selected from the group consisting of leaf, pollen, embryo, bulb, anther, flower, bud, and meristem.

Also provided herein is seed of I37554A or B, a sample of said seed having been deposited under Accession No. PTA-9054 and PTA-9055, respectively, an onion plant grown from any one of said seed, an onion plant part from any one of said onion plants, such as pollen, protoplast, an ovule, or a cell. Also provided herein is a tissue culture of cells obtained from any one of said plants, such as a tissue culture of cells from a tissue selected from the group consisting of leaf, pollen, embryo, bulb, anther, flower, bud, and meristem.

Further provided herein is a hybrid onion plant having a bulb comprising a PAD measurement of less than about 5.5 μM/g FW pyruvate, preferably less than or equal to 3.5 μM/g FW pyruvate.

Also provided herein is a long-day onion plant producing bulbs which have a mean PAD measurement at harvest of less than 3.75 μM/g fresh weight (FW) pyruvate, or equal to or less than 3.5 μM/g FW pyruvate, such as any one of said onion plants, wherein said bulbs have a mean soluble solids content (SSC) at harvest of at least 7.5%, or at least 8%.

Further provided herein is any of the above onion plants, wherein said PAD measurement is increased by less than 10% after storage for at least 4 months compared to the PAD measurement at harvest, and such or any of the above onion plants, wherein said SSC is reduced by less than 2% after storage for at least 4 months.

Also provided herein is any one of the above onion plants producing bulbs wherein the pungency level of the most pungent bulb and least pungent bulb differ by at most 5 μMol/g FW, or by at most 3.5 μMol/g FW, or such or any one of the above onion plants producing bulbs wherein all bulbs have a pungency between 0 and 5 μMol/g FW, or between 1 and 4 μMol/g FW.

Further provided herein is any of the above onion plants, wherein said onion plant requires 14 or more contiguous hours of light for 2 or more days to initiate bulb formation, such as such onion plant or any one of the above onion plants of the invention, wherein said mean PAD or mean SSC is obtained from at least 10 onion bulbs of said plant.

Also provided in accordance with the invention is any one of the above onion plants, wherein said plant is a hybrid, or is a plant derivable or obtainable from a line designated I37853B, I37554A, or I37554B, deposited under Accession Nos. PTA-9053, PTA-9054 and PTA-9055, respectively.

Further provided herein are seeds or bulbs of any one of the above onion plants, and a container comprising a plurality of such or any one of the above bulbs, such as a any such container, wherein at least 75% of the bulbs are bulbs according to claim 12. Also provided herein is any one of the above containers, wherein said container comprises at least 1 pound of bulbs according to claim 12.

Also set forth herein is a part of any of the above onion plants, or of any one of the above seeds or bulbs, such as a part a cell or cell culture, a tissue culture, a protoplast or a plant organ.

Hence, in one aspect, the invention provides long day onion plants which produce bulbs having low pungency but high SSC and/or which can be stored for at least 2, 3, 4, 5, 6, 7 months or more without any significant increase in pungency (compared to the level at harvest) and/or without any significant reduction in SSC (compared to the level at harvest). It is a further object to provide a plurality of long day plants, seeds from these plants, bulbs and containers with any of these and methods of making long day onion plants having these phenotypic characteristics.

In another aspect, the invention provides an onion plant requiring 14 or more contiguous hours of daylight to initiate bulb formation comprising a bulb having low pungency. In another aspect, the invention provides an onion plant requiring 14 or more contiguous hours of light for 2, 4, 7 or more days to initiate bulb formation. The invention provides for yellow, Spanish and other types of onion plants. The invention also provides for cells, protoplasts and tissue cultures from the plants (or plant cells) of the invention.

In a further aspect, the bulb has a PAD measurement at harvest of less than 5.5, 5.0, 4.5, 4.0, 3.8, 3.75 or 3.5 μM/g FW pyruvate. In another aspect, the bulb has a PAD measurement at harvest of 3.5 μM/g FW pyruvate, or less. In another aspect, the bulb is low pungent at harvest. In another aspect, the bulb substantially maintains low pungency after storage for about 2, 4 or 6 months. In another aspect, the PAD measurement after storage for 2, 4 or 6 months is increased less than 10% from a PAD measurement at harvest.

hi another aspect, the invention provides a part of an onion plant requiring 14 or more contiguous hours of light to initiate bulb formation, wherein said plant comprises a bulb having a PAD measurement of less than 5.5 μM/g FW pyruvate, preferably less than or equal to 3.5 μM/g FW pyruvate. The plant part may be a seed, bulb, leaf, pollen or an ovule.

In another aspect, the invention provides a container of onion bulbs from onion plants requiring 14 or more contiguous hours of light to initiate bulb formation comprising an average PAD measurement of less than about 5.5, 5.0, 4.5, 4.0, 3.75 or 3.5 μM/g FW pyruvate, or equal to 3.5 μM/g FW pyruvate. In another aspect, the invention provides that at least 75, 85 or 95% of onion bulbs in a container have a PAD measurement of less than about 5.5 μM/g FW pyruvate.

In another aspect, the invention provides a seed of an onion plant requiring 14 or more contiguous hours of light to initiate bulb formation, wherein said seed is capable of producing an onion plant having a bulb comprising a PAD measurement of less than about 5.5, 5.0, 4.5, 4.0, 3.75 or 3.5 μM/g FW pyruvate, or equal to 3.5 μM/g FW pyruvate. The invention also provides for a container of seeds, wherein onion bulbs from greater than 50% of said seeds are low pungency onions.

In another aspect, the invention provides a method of producing a hybrid onion seed comprising: crossing a low pungency onion plant requiring 14 or more hours of light to initiate bulb formation with another onion plant; and obtaining F1 onion seed, hi another aspect, the invention provides low pungency onion lines designated 137853 or 137554, seeds from these onion lines, plants grown from these seeds and plant parts and tissues from these plants.

In another aspect, the invention provides a hybrid onion plant having a bulb comprising a PAD measurement of less than about 5.5 or 3.5 μM/g FW pyruvate, or equal to 3.5 μM/g FW pyruvate.

In another aspect, the invention provides onions having high SSC, good storage ability and low pungency.

In another aspect, the invention provides an Allium cepa plant, or plant part or seed thereof, comprising in its genome one or more of QTL1 on chromosome 1, QTL2 on chromosome 2 and/or QTL7 on chromosome 7, wherein said QTL1, QTL2 and QTL7 confer a reduced pyruvate level,

wherein QTL1 is comprised in an introgression fragment comprising a haplotype of one or more of the following SNP markers:
SNP_16 comprising an Adenine at nucleotide 51 of SEQ ID NO: 31 or at nucleotide 51 of a sequence comprising at least 97% identity to SEQ ID NO: 31;
SNP_17 comprising an Adenine at nucleotide 51 of SEQ ID NO: 33 or at nucleotide 51 of a sequence comprising at least 97% identity to SEQ ID NO: 33;
SNP_05 comprising a Cytosine at nucleotide 51 of SEQ ID NO: 9 or at nucleotide 51 of a sequence comprising at least 97% identity to SEQ ID NO: 9;
SNP_06 comprising an Adenine at nucleotide 51 of SEQ ID NO: 11 or at nucleotide 51 of a sequence comprising at least 97% identity to SEQ ID NO: 11;
SNP_07 comprising a Cytosine at nucleotide 51 of SEQ ID NO: 13 or at nucleotide 51 of a sequence comprising at least 97% identity to SEQ ID NO: 13;
SNP_08 comprising a Thymine at nucleotide 51 of SEQ ID NO: 15 or at nucleotide 51 of a sequence comprising at least 97% identity to SEQ ID NO: 15;
SNP_18 comprising a Thymine at nucleotide 51 of SEQ ID NO: 35 or at nucleotide 51 of a sequence comprising at least 97% identity to SEQ ID NO: 35;
SNP_19 comprising a Thymine at nucleotide 51 of SEQ ID NO: 37 or at nucleotide 51 of a sequence comprising at least 97% identity to SEQ ID NO: 37;
SNP_20 comprising a Guanine at nucleotide 51 of SEQ ID NO: 39 or at nucleotide 51 of a sequence comprising at least 97% identity to SEQ ID NO: 39; and/or
SNP_21 comprising a Cytosine at nucleotide 51 of SEQ ID NO: 41 or at nucleotide 51 of a sequence comprising at least 97% identity to SEQ ID NO: 41,

wherein QTL2 is comprised in an introgression fragment comprising a haplotype of one or more of the following SNP markers:
SNP_11 comprising an Adenine at nucleotide 51 of SEQ ID NO: 21 or at nucleotide 51 of a sequence comprising at least 97% identity to SEQ ID NO: 21;
SNP_12 comprising a Cytosine at nucleotide 51 of SEQ ID NO: 23 or at nucleotide 51 of a sequence comprising at least 97% identity to SEQ ID NO: 23;
SNP_13 comprising a Thymine at nucleotide 51 of SEQ ID NO: 25 or at nucleotide 51 of a sequence comprising at least 97% identity to SEQ ID NO: 25;
SNP_14 comprising an Adenine at nucleotide 51 of SEQ ID NO: 27 or at nucleotide 51 of a sequence comprising at least 97% identity to SEQ ID NO: 27;
SNP_01 comprising a Thymine at nucleotide 51 of SEQ ID NO: 1 or at nucleotide 51 of a sequence comprising at least 97% identity to SEQ ID NO: 1;
SNP_02 comprising an Adenine at nucleotide 51 of SEQ ID NO: 3 or at nucleotide 51 of a sequence comprising at least 97% identity to SEQ ID NO: 3;
SNP_03 comprising a Cytosine at nucleotide 51 of SEQ ID NO: 5 or at nucleotide 51 of a sequence comprising at least 97% identity to SEQ ID NO: 5;
SNP_04 comprising a Thymine at nucleotide 51 of SEQ ID NO: 7 or at nucleotide 51 of a sequence comprising at least 97% identity to SEQ ID NO: 7; and/or
SNP_15 comprising a Guanine at nucleotide 51 of SEQ ID NO: 29 or at nucleotide 51 of a sequence comprising at least 97% identity to SEQ ID NO: 29, and/or

wherein QTL7 is comprised in an introgression fragment comprising a haplotype of one or more of the following SNP markers:
SNP_22 comprising a Thymine at nucleotide 51 of SEQ ID NO: 43 or at nucleotide 51 of a sequence comprising at least 97% identity to SEQ ID NO: 43;
SNP_23 comprising a Thymine at nucleotide 51 of SEQ ID NO: 45 or at nucleotide 51 of a sequence comprising at least 97% identity to SEQ ID NO: 45;
SNP_24 comprising a Cytosine at nucleotide 51 of SEQ ID NO: 47 or at nucleotide 51 of a sequence comprising at least 97% identity to SEQ ID NO: 47;
SNP_25 comprising a Guanine at nucleotide 51 of SEQ ID NO: 49 or at nucleotide 51 of a sequence comprising at least 97% identity to SEQ ID NO: 49;
SNP_09 comprising a Thymine at nucleotide 51 of SEQ ID NO: 17 or at nucleotide 51 of a sequence comprising at least 97% identity to SEQ ID NO: 17; and/or
SNP_10 comprising a Guanine at nucleotide 51 of SEQ ID NO: 19 or at nucleotide 51 of a sequence comprising at least 97% identity to SEQ ID NO: 19.

In another aspect, the plant comprises two or more of QTL1 on chromosome 1, QTL2 on chromosome 2 and/or QTL7 on chromosome 7. In other aspects, QTL1 is comprised in an introgression fragment comprising a haplotype of two, three, four, five, or more of the following SNP markers: SNP_16, SNP_17, SNP_05, SNP_06, SNP_07, SNP_08, SNP_18, SNP_19, SNP_20 and/or SNP_21. In other aspects, QTL2 is comprised in an introgression fragment comprising a haplotype of two, three, four, five or more of the following SNP markers: SNP_11, SNP_12, SNP_13, SNP_14, SNP_01, SNP_02, SNP_03, SNP_04 and/or SNP_15. In other aspects, QTL7 is comprised in an introgression fragment comprising a haplotype of two, three, four, five, or more of the following SNP markers: SNP_22, SNP_23, SNP_24, SNP_25, SNP_09 and/or SNP_10. In other aspects, the plant is a single cross F1 hybrid or an inbred line.

In another aspect, the invention provides a method of producing an Allium cepa plant comprising in its genome one or more of QTL1 on chromosome 1, QTL2 on chromosome 2 and/or QTL7 on chromosome 7, wherein said QTL1, QTL2 and QTL7 confer a reduced pyruvate level, said method comprising: a) crossing a first onion plant comprising in its genome one or more of QTL1, QTL2 and/or QTL7 with a second onion plant, and b) collecting seeds from said cross, wherein QTL1, QTL2 and/or QTL7 are comprised in introgression fragments comprising a haplotype of one or more of the following SNP markers as described herein.

### BRIEF DESCRIPTION OF THE SEQUENCE LISTING

The accompanying sequence listing filed herewith, with reference number 039621.00519, is incorporated by reference herein.

SEQ ID NO: 1 shows the reduced pungency genotype of SNP_01.

SEQ ID NO: 2 shows the high pungency genotype of SNP_01.

SEQ ID NO: 3 shows the reduced pungency genotype of SNP_02.

SEQ ID NO: 4 shows the high pungency genotype of SNP_02.

SEQ ID NO: 5 shows the reduced pungency genotype of SNP_03.

SEQ ID NO: 6 shows the high pungency genotype of SNP_03.

SEQ ID NO: 7 shows the reduced pungency genotype of SNP_04.

SEQ ID NO: 8 shows the high pungency genotype of SNP_04.

SEQ ID NO: 9 shows the reduced pungency genotype of SNP_05.

SEQ ID NO: 10 shows the high pungency genotype of SNP_05.

SEQ ID NO: 11 shows the reduced pungency genotype of SNP_06.

SEQ ID NO: 12 shows the high pungency genotype of SNP_06.

SEQ ID NO: 13 shows the reduced pungency genotype of SNP_07.

SEQ ID NO: 14 shows the high pungency genotype of SNP_07.

SEQ ID NO: 15 shows the reduced pungency genotype of SNP_08.

SEQ ID NO: 16 shows the high pungency genotype of SNP_08.

SEQ ID NO: 17 shows the reduced pungency genotype of SNP_09.

SEQ ID NO: 18 shows the high pungency genotype of SNP_09.

SEQ ID NO: 19 shows the reduced pungency genotype of SNP_10.

SEQ ID NO: 20 shows the high pungency genotype of SNP_10.

SEQ ID NO: 21 shows the reduced pungency genotype of SNP_11.

SEQ ID NO: 22 shows the high pungency genotype of SNP_11.

SEQ ID NO: 23 shows the reduced pungency genotype of SNP_12.

SEQ ID NO: 24 shows the high pungency genotype of SNP_12.

SEQ ID NO: 25 shows the reduced pungency genotype of SNP_13.

SEQ ID NO: 26 shows the high pungency genotype of SNP_13.

SEQ ID NO: 27 shows the reduced pungency genotype of SNP_14.

SEQ ID NO: 28 shows the high pungency genotype of SNP_14.

SEQ ID NO: 29 shows the reduced pungency genotype of SNP_15.

SEQ ID NO: 30 shows the high pungency genotype of SNP_15.

SEQ ID NO: 31 shows the reduced pungency genotype of SNP_16.

SEQ ID NO: 32 shows the high pungency genotype of SNP_16.

SEQ ID NO: 33 shows the reduced pungency genotype of SNP_17.

SEQ ID NO: 34 shows the high pungency genotype of SNP_17.

SEQ ID NO: 35 shows the reduced pungency genotype of SNP_18.

SEQ ID NO: 36 shows the high pungency genotype of SNP_18.

SEQ ID NO: 37 shows the reduced pungency genotype of SNP_19.

SEQ ID NO: 38 shows the high pungency genotype of SNP_19.

SEQ ID NO: 39 shows the reduced pungency genotype of SNP_20.

SEQ ID NO: 40 shows the high pungency genotype of SNP_20.

SEQ ID NO: 41 shows the reduced pungency genotype of SNP_21.

SEQ ID NO: 42 shows the high pungency genotype of SNP_21.

SEQ ID NO: 43 shows the reduced pungency genotype of SNP_22.

SEQ ID NO: 44 shows the high pungency genotype of SNP_22.

SEQ ID NO: 45 shows the reduced pungency genotype of SNP_23.

SEQ ID NO: 46 shows the high pungency genotype of SNP_23.

SEQ ID NO: 47 shows the reduced pungency genotype of SNP_24.

SEQ ID NO: 48 shows the high pungency genotype of SNP_24.

SEQ ID NO: 49 shows the reduced pungency genotype of SNP_25.

SEQ ID NO: 50 shows the high pungency genotype of SNP_25.

### DETAILED DESCRIPTION OF THE INVENTION

### Definitions

“Phenotype” is the observable external and/or physiological appearance of the plant as a result of the interaction between its genotype and its environment. It includes all observable morphological and physiological characteristics and thus encompasses phenotypes such as pungency, PAD measurements and soluble solid contents of onion bulbs.

“Genotype” is the total of inheritable genetic information of a plant, partly influenced by the environmental factors, which is expressed in the phenotype.

As used herein, “Onion plant” or “onion” is a plant of the botanical species Allium cepa L. or parts thereof, such as the (harvested) bulb, seeds, etc. “Bulb” is the harvested, edible portion of the plant. Onion bulbs may be developing or mature. Herein mature bulbs are preferred, which are bulbs ready for harvest or harvested.

“Long-day” onion plants will initiate bulb formation when light (day length) is at least about 14 contiguous hours or more, e.g., at least about 14, 15 or 16 hours. Preferably this contiguous light (hours per day) is provided for 2, 4, 7, 14, 21, 25 or more days to initiate bulb formation.

“Storage conditions” encompass typical conditions used to store (preferably fresh) onions, such as darkness, cool temperature (as used herein, a cool temperature means preferably below 12° C., e.g., about 3-12° C., 3-10° C., 5-10° C. or about 3-5° C., preferably about 3, 4 or 5 degrees Celsius) and a relative humidity (RH) of about 60-80%, preferably about 70-80%, most preferably around 70%. Also preferred is controlled ventilation.

A “family” is the progeny of one plant, which has been pollinated by a number of different other plants.

“Hybrid” or “hybrid plant” is a plant produced by the inter-crossing (cross-fertilization) of at least two different plants or plants of different parent lines. It is understood that the seeds of such a cross (hybrid seeds) are encompassed herein, as well as the hybrid plants grown from those seeds and plant parts derived from those grown plants (e.g. bulbs).

“F1, F2, etc.” refers to the consecutive related generations following a cross between two parent plants or parent lines. The plants grown from the seeds produced by crossing two plants or lines is called the F1 generation. Selling the F1 plants results in the F2 generation, etc.

“Soluble Solids” or “Soluble Solids Content” (“SSC” herein), is the percentage (%) of water-soluble compounds in onion bulbs as measured by a refractometer according to the method of Mann and Hoyle, 1945 (Proc. Americ. Soc. Hort. Sci. 46: 285-292) or Foskett and Peterson, 1949 (Proc. Americ. Soc. Hort. Sci. 55: 314-318).

“High SSC” refers herein to an average SSC of a representative number of onion bulbs (e.g., at least 10, 15, 20, 30, 40, 50, 50, 60, 70, 80, 90 or more bulbs) of at least 7% or 7.5%, preferably at least 8%, 9%, 10%, 11%, 12%, 15%, 20%, 25%, 30% or more. Thus, average SSC of 7.0-30%, 7.5-30%, or even 7.0-20%, 8.0-20%, 7.0-15%, 8.0-15%, 7.0-10%, etc. are encompassed herein.

“Pungency” is the typical sharp taste of onion as the onion bulb tissue disintegrates by comminution. Pungency is preferably determined by measuring the enzymatic development of pyruvic acid according to the method of Schwimmer and Weston (1961, J. of Agric. Food Chemistry 9:301-304), which is strongly correlated to the flavour perception by a test panel (Schwimmer 1962, J. Food Sci. 27: 94-97; Wall and Corgan, 1992, Hort. Science 27: 1029-1030). Pungency is expressed as μMol (micromoles, also μM or μmol herein) per gram fresh weight bulb material (μMol/g FW). It is also referred to as “PAD measurement” (PAD from Pyruvic Acid Development) or “pyruvate measurement” or “pyruvate level” herein.

[73] The term “reduced pungency” as used herein accordingly refers to a pungency level that is reduced when compared to the pungency level of a reference variety. Preferably, the reduced pungency level corresponds to a pungency level that is reduced to such an extent that the reduced pungency level corresponds to a low pungency level. “Low pungency” refers herein to an average pungency of a representative number of (mature) onion bulbs (e.g., at least about 5, 8, 10, 15, 20, 30, 40, 50, 50, 60, 70, 80, 90 or more bulbs) of less than 5.5 μMol/g FW, preferably less than 5.0, 4.5, 4.0 μMol/g FW, more preferably equal to or less than 3.8 or 3.75 μMol/g FW, most preferably equal to or less than 3.5, 3.0, 2.5, 2.3, 2.0, 1.8, 1.5, or 1.3 μMol/g FW, as determined by PAD measurement. Thus, average pungencies of between 3.5 and 1.3 μMol/g FW, between 3.0 and 1.3 μMol/g FW, or between 3.0 and 2.0 μMol/g FW, etc. are encompassed herein. “High pungency” refers herein to an average pungency level of a representative number of (mature) onion bulbs that is higher than the low pungency level as defined herein, preferably more than 5.5 μMol/g FW, or even more than 6.0, 6.5, or 7.0 μMol/g FW. Pungency can be measured at harvest and/or after 2, 3, 4, 5, 6, 7, 8 or more months of storage.

A “narrow pungency range” refers to the variance in pungency between individual bulbs of a plurality of bulbs obtained from one plant line being narrow, i.e., the pungency level of the most pungent bulb (maximum value) and least pungent bulb (minimum value) differ preferably by less than or at most 5 μMol/g FW, more preferably less than or at most 4 μMol/g FW or less than or at most 3.5 μMol/g FW, more preferably by less than or at most 3.0, 2.5, 2.0, 1.5 or 1.0 μMol/g FW. Preferably the maximum pungency (of the most pungent bulb produced by the plant) is equal to or less than 5 μMol/g FW, preferably equal to or less than 4.9, 4.8, 4.75, 4.7, 4.5, 4.0 or 3.8, 3.7, 3.5 or 3.0 μMol/g FW. Preferably the minimum pungency level (i.e. of the least pungent bulb produced by the plant) is equal to or below 3.0, 2.5, more preferably equal to or below 2.0, 1.3 or 1.2 μMol/g FW. Preferred ranges of pungency within a plant line are, thus, that all bulbs have a pungency between 0 (min) and 5 (max) μMol/g FW, preferably between 1 (min) and 5 (max) μMol/g FW, more preferably between 1 (min) and 4 (max) μMol/g FW. Also, in one embodiment of the invention, all bulbs have a pungency between 0 (min) and 5 (max) μMol/g FW, preferably between 1 (min) and 5 (max) μMol/g FW, more preferably between 1 or 1.2 (min) and 4.9, 4.8, 4.7 or 4.5 (max) μMol/g FW, more preferably between 1 (min) and 4 (max) μMol/g FW. A narrow pungency range is an important quality characteristic for the consumer. It can be measured at harvest and/or, preferably, after a certain period of storage, e.g. after at least about 2, 3, 4, 5, 6, 7, 8 or more months of storage.

“High pungency allele” refers herein to an allele associated with the high pungency trait as further defined herein. In one embodiment, the high pungency allele is a wild type allele.

“Reduced pungency allele” refers herein to an allele associated with the reduced pungency trait as further defined herein. In one embodiment, the reduced pungency allele is a mutant allele.

“Wild type plant” refers herein to a plant of the species Allium cepa producing bulbs having a high pungency as defined herein. Such plants are for example suitable controls in phenotypic essays, particularly if said control plants have the same genetic background as the plants (e.g., low pungency plants) that are subjected to phenotypic testing.

“Long storage” refers herein to a storage length of at least 2, 3, 4, 5, 6, 7 or more months. Preferably, there is no significant increase in pungency and/or no significant reduction in SSC during the storage period, i.e., when comparing the average pungency and/or SSC at harvest (or shortly after harvest) with the pungency and/or SSC level after 2, 3, 4, 5, 6, 7 or more months of storage. “No significant increase in pungency” refers herein to an increase in pungency measurement (i.e., pyruvate) after the storage period by less than 10%, more preferably less than 5%, even more preferably less than 3%, 2% or 1%, more preferably no increase at all, and optionally even a reduction in pungency, compared to the measurement at harvest (or shortly after harvest). “No significant reduction in SSC” refers herein to a reduction in SSC levels after the storage period of less than 5%, 4%, 3% or 2%, preferably less than 1% or 0.5%, more preferably unchanged, compared to the SSC level at harvest (or shortly after harvest). In one embodiment the mean SSC level after 2, 3, 4, 5, 6, 7 or more months of storage is at least about 80%, 85%, 87%, 88%, 89%, 90%, 95%, 98% of the level at harvest, more preferably at least about 100%, or 101%, 102%, 103%, 105% of the level at harvest, or more.

A genetic determinant can be inherited in a recessive manner, an intermediate manner, or in a dominant manner. Selection for the phenotypic trait is easier when intermediate or dominant inheritance is involved, as a larger part of the progeny of a cross reveals the trait. In general, a genetic determinant can also comprise a combination of recessive and/or intermediate and/or dominant genes or QTLs.

Selection for a genetic determinant (e.g. a reduced pungency allele) can be done on phenotype (the trait that can be observed). Selection can also be done by using molecular genotyping methods, such as one or more molecular markers that are genetically linked to the reduced pungency allele or preferably using the gene or allele sequence itself, e.g. by molecular methods which are able to distinguish between the presence of a reduced pungency allele and wild type allele, or products thereof (such as mRNA or protein encoded by the allele). The use of molecular genotyping methods in breeding (such as “marker assisted selection” when genetically linked markers are used, or other genotyping methods, such as SNP genotyping) requires a smaller population for screening (when compared to phenotypical selection) and can be done in a very early stage. A further advantage of molecular genotyping methods is the possibility to easily distinguish between homozygous plants or seeds having no copies of any of the high pungency alleles as described herein from plants having one or more copies of one or more of said high pungency alleles, which can be done even before seeds germinate or in early plant development, e.g. before onion bulbs have developed.

A “plant line” or “breeding line” refers to a plant and its progeny. As used herein, the term “inbred line” refers to a plant line which has been repeatedly selfed and is nearly homozygous for every characteristic. Thus, an “inbred line” or “parent line” refers to a plant which has undergone several generations (e.g. at least 4, 5, 6, 7 or more) of inbreeding, resulting in a plant line with a high uniformity.

The term “allele(s)” means any of one or more alternative forms of a DNA sequence at a particular locus, all of which alleles relate to one trait or characteristic at a specific locus. In a diploid cell of an organism, alleles of a given gene are located at a specific location, or locus (loci plural) on a chromosome. One allele is present on each chromosome of the pair of homologous chromosomes. A diploid plant species may comprise a large number of different alleles at a particular locus. These may be identical alleles of the gene (homozygous) or two different alleles (heterozygous).

The term “locus” (plural loci) means a specific place or places or a site on a chromosome where for example a gene or genetic marker is found. The reduced pungency loci as described herein thus are the locations in the genome of an Allium cepa plant where the reduced pungency alleles are found.

The term “linkage group” as used herein is defined as a group of loci that are physically linked together on a single molecule of DNA (a chromosome) and are more often transmitted to progeny together than would be expected according to the law of independent assortment. In the present invention, eight linkage groups were identified. Preferably, each of the eight linkage groups as used herein corresponds to one of the eight chromosomes of the onion genome.

The term “gene” means a (genomic) DNA sequence comprising a region (transcribed region), which is transcribed into a messenger RNA molecule (mRNA) in a cell, and an operably linked regulatory region (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites. Different alleles of a gene are thus different alternative forms of the gene, which may be in the form of e.g. differences in one or more nucleotides of the genomic DNA sequence (e.g. in the promoter sequence, the exon sequences, intron sequences, etc.), mRNA and/or amino acid sequence of the encoded protein. A gene may be an endogenous gene (in the species of origin) or a chimeric gene (e.g. a transgene or cis-gene). The “promoter” of a gene sequence is defined as a region of DNA that initiates transcription of a particular gene. Promoters are located near the genes they transcribe, on the same strand and upstream on the DNA. Promoters can be about 100-1000 base pairs long. In one aspect the promoter is defined as the region of about 1000 base pairs or more e.g. about 1500 or 2000, upstream of the start codon (i.e. ATG) of the protein encoded by the gene.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). The coding sequence may be in sense-orientation and encodes a desired, biologically active protein or peptide, or an active peptide fragment.

A “quantitative trait locus”, or “QTL” is a chromosomal locus that encodes for one or more alleles that affect the expressivity of a continuously distributed (quantitative) phenotype.

“Physical distance” between loci (e.g. between molecular markers and/or between phenotypic markers) on the same chromosome is the actual physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

“Introgression fragment” or “introgression segment” or “introgression region” refers to a chromosome fragment (or chromosome part or region) which has been introduced into another plant of the same or related species by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three-quarters or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

The term “isogenic plant” refers to two plants which are genetically identical except for the reduced pungency allele of the present invention. In order to investigate the impact of the reduced pungency trait, one can cross a plant line (or variety) of interest with a plant comprising the reduced pungency allele reduced pungency trait and select for progeny expressing the desired trait. Optionally one may have to self the progeny one or more times to be able to determine the genetic determinants for the reduced pungency trait in the plant phenotype. Said progeny can then be backcrossed (at least 2 times, e.g. 3, 4, or preferably 5 or 6 times) with the plant line (or variety) of interest while selecting for progeny having the same phenotype as the plant line (or variety) of interest and expressing the genetic determinants for the reduced pungency trait. The impact of the reduced pungency can then be compared between the plant line (variety) of interest and its isogenic line not comprising the genetic determinants for the reduced pungency trait.

The term “nucleic acid”, “nucleic acid sequence”, “nucleic acid molecule” or “polynucleotide” are used interchangeably and refer to a DNA or RNA molecule in single or double stranded form, particularly a DNA encoding a protein or protein fragment according to the invention. An “isolated nucleic acid” refers to a nucleic acid which is no longer in the natural environment from which it was isolated, e.g. the nucleic acid in a bacterial host cell or in the plant nuclear or plastid genome.

The terms “protein”, “peptide sequence”, “amino acid sequence” or “polypeptide” are used interchangeably and refer to molecules consisting of a chain of amino acids, without reference to a specific mode of action, size, 3-dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

An “active protein” or “functional protein” is a protein which has protein activity as measurable in vitro, e.g. by an in vitro activity assay, and/or in vivo, e.g. by the phenotype conferred by the protein. A “wild type” protein is a fully functional protein, as present in the wild type plant. A “mutant protein” is herein a protein comprising one or more mutations in the nucleic acid sequence encoding the protein, whereby the mutation results in (the mutant nucleic acid molecule encoding) a protein having altered activity, preferably a protein having reduced activity, most preferably a protein having no activity.

“Functional derivatives” of a protein as described herein are fragments, variants, analogues, or chemical derivatives of the protein which retain at least a portion of the activity or immunological cross reactivity with an antibody specific for the mutant protein.

A fragment of a mutant protein refers to any subset of the molecule.

Variant peptides may be made by direct chemical synthesis, for example, using methods well known in the art.

An analogue of a mutant protein refers to a non-natural protein substantially similar to either the entire protein or a fragment thereof.

A “mutation” in a nucleic acid molecule is a change of one or more nucleotides compared to the wild type sequence, e.g. by replacement, deletion or insertion of one or more nucleotides.

A “mutation” in an amino acid molecule making up a protein is a change of one or more amino acids compared to the wild type sequence, e.g. by replacement, deletion or insertion of one or more amino acids. Such a protein is then also referred to as a “mutant protein”.

A “point mutation” is the replacement of a single nucleotide, or the insertion or deletion of a single nucleotide.

A “nonsense mutation” is a (point) mutation in a nucleic acid sequence encoding a protein, whereby a codon in a nucleic acid molecule is changed into a stop codon. This results in a pre-mature stop codon being present in the mRNA and results in translation of a truncated protein. A truncated protein may have decreased function or loss of function.

A “missense or non-synonymous mutation” is a (point) mutation in a nucleic acid sequence encoding a protein, whereby a codon is changed to code for a different amino acid. The resulting protein may have decreased function or loss of function.

A “splice-site mutation” is a mutation in a nucleic acid sequence encoding a protein, whereby RNA splicing of the pre-mRNA is changed, resulting in an mRNA having a different nucleotide sequence and a protein having a different amino acid sequence than the wild type. The resulting protein may have decreased function or loss of function.

A “frame shift mutation” is a mutation in a nucleic acid sequence encoding a protein by which the reading frame of the mRNA is changed, resulting in a different amino acid sequence. The resulting protein may have decreased function or loss of function.

A “deletion” in context of the invention shall mean that anywhere in a given nucleic acid sequence at least one nucleotide is missing compared to the nucleic sequence of the corresponding wild type sequence or anywhere in a given amino acid sequence at least one amino acid is missing compared to the amino acid sequence of the corresponding (wild type) sequence.

A “truncation” shall be understood to mean that at least one nucleotide at either the 3′-end or the 5′-end of the nucleotide sequence is missing compared to the nucleic sequence of the corresponding wild type sequence or that at least one amino acid at either the N-terminus or the C-terminus of the protein is missing compared to the amino acid sequence of the corresponding wild type protein, whereby in a 3′-end or C-terminal truncation at least the first nucleotide at the 5′-end or the first amino acid at the N-terminus, respectively, is still present and in a 5′-end or N-terminal truncation at least the last nucleotide at the 3′-end or the last amino acid at the C-terminus, respectively, is still present. The 5′-end is determined by the ATG codon used as start codon in translation of a corresponding wild type nucleic acid sequence.

“Replacement” shall mean that at least one nucleotide in a nucleic acid sequence or one amino acid in a protein sequence is different compared to the corresponding wild type nucleic acid sequence or the corresponding wild type amino acid sequence, respectively, due to an exchange of a nucleotide in the coding sequence of the respective protein.

“Insertion” shall mean that the nucleic acid sequence or the amino acid sequence of a protein comprises at least one additional nucleotide or amino acid compared to the corresponding wild type nucleic acid sequence or the corresponding wild type amino acid sequence, respectively.

“Pre-mature stop codon” in context with the present invention means that a stop codon is present in a coding sequence (cds) which is closer to the start codon at the 5′-end compared to the stop codon of a corresponding wild type coding sequence.

A “mutation in a regulatory sequence”, e.g. in a promoter or enhancer of a gene, is a change of one or more nucleotides compared to the wild type sequence, e.g. by replacement, deletion or insertion of one or more nucleotides, leading for example to decreased or no mRNA transcript of the gene being made. The “promoter of a gene sequence”, accordingly is defined as a region of DNA that initiates transcription of a particular gene. Promoters are located near the genes they transcribe, on the same strand and upstream on the DNA. Promoters can be about 100-1000 base pairs long. In one aspect, the promoter is defined as the region of about 2000 base pairs or more upstream of the start codon (i.e. ATG) of the protein encoded by the gene, preferably, the promoter is the region of about 1500 base pairs upstream of the start codon, more preferably the promoter is the region of about 1000 base pairs upstream of the start codon.

As used herein, the term “operably linked” refers to a linkage of polynucleotide elements in a functional relationship. A nucleic acid is “operably linked” when it is placed into a functional relationship with another nucleic acid sequence. For instance, a promoter, or rather a transcription regulatory sequence, is operably linked to a coding sequence if it affects the transcription of the coding sequence. Operably linked means that the nucleic acid sequences being linked are typically contiguous.

A “fragment” of the gene or DNA sequence refers to any subset of the molecule, e.g., a shorter polynucleotide or oligonucleotide. In one aspect the fragment comprises the mutation as defined by the invention.

A “variant” of the gene or DNA refers to a molecule substantially similar to either the entire gene or a fragment thereof, such as a nucleotide substitution variant having one or more substituted nucleotides, but which maintains the ability to hybridize with the particular gene or to encode mRNA transcript which hybridizes with the native DNA. Preferably the variant comprises the reduced pungency allele as defined by the invention.

As used herein, the term “plant” includes the whole plant or any parts or derivatives thereof, such as plant organs (e.g., harvested or non-harvested flowers, leaves, bulbs, etc.), plant cells, plant protoplasts, plant cell or tissue cultures from which whole plants can be regenerated, regenerable or non-regenerable plant cells, plant calli, plant cell clumps, and plant cells that are intact in plants, or parts of plants, such as embryos, pollen, ovules, ovaries (e.g., harvested tissues or organs), flowers, leaves, seeds, bulbs, clonally propagated plants, roots, stems, cotyledons, hypocotyls, root tips and the like. Also any developmental stage is included, such as seedlings, immature and mature, etc. Preferably, the plant part or derivative comprises the gene or locus as defined by the current invention.

A “plant line” or “breeding line” refers to a plant and its progeny.

“Plant variety” or “variety” is a group of plants within the same botanical taxon of the lowest grade known, which (irrespective of whether the conditions for the recognition of plant breeder's rights are fulfilled or not) can be defined on the basis of the expression of characteristics that result from a certain genotype or a combination of genotypes, can be distinguished from any other group of plants by the expression of at least one of those characteristics, and can be regarded as an entity, because it can be multiplied without any change. Therefore, the term “plant variety” cannot be used to denote a group of plants, even if they are of the same kind, if they are all characterized by the presence of 1 locus or gene (or a series of phenotypical characteristics due to this single locus or gene), but which can otherwise differ from one another enormously as regards the other loci or genes. “F1, F2, etc.” refers to the consecutive related generations following a cross between two parent plants or parent lines. The plants grown from the seeds produced by crossing two plants or lines is called the F1 generation. Selfing the F1 plants results in the F2 generation, etc. “F1 hybrid” plant (or F1 seed, or hybrid) is the generation obtained from crossing two inbred parent lines. “Selfing”, accordingly, refers to the self-pollination of a plant, i.e. to the union of gametes from the same plant.

“Backcrossing” refers to a breeding method by which a (single) trait, such as the capability for inducing a decreased pungency, can be transferred from one genetic background (also referred to as “donor” generally, but not necessarily, this is an inferior genetic background) into another genetic background (also referred to as “recurrent parent”; generally, but not necessarily, this is a superior genetic background). An offspring of a cross (e.g. an F1 plant obtained by crossing a first plant of a certain plant species comprising the reduced pungency allele of the present invention with a second plant of the same plant species or of a different plant species that can be crossed with said first plant species wherein said second plant species does not comprise the reduced pungency allele of the present invention; or an F2 plant or F3 plant, etc., obtained by selfing the F1) is “backcrossed” to a parent plant of said second plant species. After repeated backcrossing, the trait of the donor genetic background, e.g. the reduced pungency allele conferring reduced pungency trait of the present invention, will have been incorporated into the recurrent genetic background. The terms “gene converted” or “conversion plant” or “single locus conversion” in this context refer to plants which are developed by backcrossing wherein essentially all of the desired morphological and/or physiological characteristics of the recurrent parent are recovered in addition to the one or more genes transferred from the donor parent. The plants grown from the seeds produced by backcrossing of the F1 plants with the second parent plant line is referred to as the “BC1 generation”. Plants from the BC1 population may be selfed resulting in the BC1F2 generation or backcrossed again with the cultivated parent plant line to provide the BC2 generation. An “M1 population” is a plurality of mutagenized seeds/plants of a certain plant line. “M2, M3, M4, etc.” refers to the consecutive generations obtained following selfing of a first mutagenized seed/plant (M1).

The term “cultivated plant” or “cultivar” refers to plants of a given species, e.g. varieties, breeding lines or cultivars of the said species, cultivated by humans and having good agronomic characteristics. The so-called heirloom varieties or cultivars, i.e. open pollinated varieties or cultivars commonly grown during earlier periods in human history and often adapted to specific geographic regions, are in one aspect of the invention encompassed herein as cultivated plants. The term “cultivated plant” does not encompass wild plants. “Wild plants” include for example wild accessions.

The term “food” is any substance consumed to provide nutritional support for the body. It is usually of plant or animal origin, and contains essential nutrients, such as carbohydrates, fats, proteins, vitamins, or minerals. The substance is ingested by an organism and assimilated by the organism's cells in an effort to produce energy, maintain life, or stimulate growth. The term food includes substance consumed to provide nutritional support for both the human and animal body.

Throughout this document “average” and “mean” are used interchangeably and refer to the arithmetic mean.

It is understood that comparisons between different plant lines involves growing a number of plants of a line (or variety) (e.g. at least 5 plants, preferably at least 10 plants per line) under the same conditions as the plants of one or more control plant lines (preferably wild type plants) and the determination of differences, preferably statistically significant differences, between the plant lines when grown under the same environmental conditions. Preferably the plants are of the same line or variety.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids or nucleic acids) are referred to.

### Plants

An onion plant (and seed of an onion plant, and parts derived from such a plant) requiring 14 or more contiguous hours of daylight to initiate bulb formation is provided, whereby the bulb has a low pungency, especially at harvest. Preferably the bulb also has a high soluble solid content (SSC), especially at harvest. The bulb substantially maintains pungency after storage for about 2, 3, 4, 5, 6, 7 or more months. The bulb also substantially maintains SSC content for about 2, 3, 4, 5, 6, 7 or more months.

In one aspect, the invention provides onion plants, bulbs and seeds, whereby the bulbs comprise a low (mean) pungency at harvest, a high (mean) SSC and/or a long storage ability. The (mean) pungency at harvest is preferably less than 5.5, 5.0, 4.5, 4.0 μMol/g FW, more preferably equal to or less than 3.8 or 3.75 μMol/g FW, preferably equal to or less than 3.5, 3.0, 2.5, 2.3, 2.0, 1.8, 1.5, 1.3 μMol/g FW or less. The (mean) SSC at harvest is preferably at least 7.0%, 7.5%, or more preferably at least 8%, 9%, 10%, 11%, 12%, 15%, 20%, 25%, 30% or more. The bulbs according to the invention can be stored for at least 2, 3, 4, 5, 6, 7 or more months, preferably without any significant increase in pungency at the end of storage and/or without any significant reduction in SSC.

In addition, the onion plants provided herein produce bulbs which have a narrow pungency range as defined above. This characteristic is an important feature for the consumer of fresh onions, as often a bag of onions are bought but individual onions are used for the preparation of food. Thus, in one embodiment a plurality of (harvested) onion bulbs are provided which have a narrow pungency range, as are plants which are capable of producing such bulbs.

Also progeny of the above plants are provided (obtained by selling or crossing), which retain bulbs with a low level of pungency, high SSC content and/or long storage ability, i.e. which are substantially identical to the bulbs of the parent(s) for these traits. Progeny include thus, for example inbred plants producing bulbs with one or more of the above traits or hybrid plants producing bulbs with one or more of the above traits.

Also parts of the above onion plants are provided. Such plant parts, derived from the onion plants described herein, may be a seed, a bulb, a leaf, a flower, pollen, stamen, an ovule, a cell, a protoplast, a tissue culture of cells or the like. A tissue culture of cells may, for example, be derived from a tissue selected from a leaf, pollen, embryo, bulb, flower, anther, pollen, ovule, bud, meristem or any cell.

In one aspect, the invention relates to long-day onion seeds deposited by Nunhems B. V. at the American Type Culture Collection (ATCC, 10801 University Boulevard, Manassas, Va. 20110-2209, USA) under the Budapest Treaty under accession number PTA-9053 (seeds of line I37853B), PTA-9054 (seeds of line I37554A) and PTA-9055 (seeds of line I37554B) on Mar. 13, 2008, or any derivatives thereof, such as progeny obtained by selling any one of the deposited plants or by crossing of any one of the deposited plants with another onion plant. In one aspect, derivatives include inbred onion plants which comprise the low pungency, high SSC and/or long storage ability as described. In another aspect, derivatives include onion plants or seeds (and bulbs) obtained from using one of these lines (I37554A or B, 137853 or a derivative of any of these) as parent in one or more crosses with a further onion plant and/or one or more sellings, whereby the progeny have the same (or better) low pungency, high soluble solid phenotypes and/or storage properties as defined above and/or as the deposited lines. Therefore, derivatives may include hybrid onion plants or seeds (and bulbs of such plants) which produce/are capable of producing bulbs having the above (or better) low pungency, high SSC and/or long term storage abilities as described above and/or as the bulbs of 137853 and/or I37554A or B. Derivatives of the hybrids are also encompassed herein. In another aspect, hybrid seeds, plants and bulbs obtainable from crossing 137853 (or a derivative thereof, such as an inbred) with I37554A or B (or a derivative thereof, such as an inbred) are provided, as well as plants, bulbs and seeds obtained from using such F1 hybrids in further selfings or crosses. Therefore, various long day onion plants having low pungency, high SSC and/or long term storage ability are encompassed herein, including, for example, plants comprising the physiological and morphological characteristics of 137853 and/or I37554A or B.

Thus, a long day onion plant derived from, or derivable from, one of the plants deposited under Accession number PTA-9053, PTA-9054 or PTA-9055 by selfing, crossing, clonal propagation, or tissue culture is provided herein, wherein the plant produces onion bulbs having the same (or better) low pungency, high soluble solid phenotypes and/or storage properties (at harvest and/or after storage) as described herein and/or as the deposited lines PTA-9053, PTA-9054 or PTA-9055.

Progeny of the onion plants deposited under PTA-9053, PTA-9054 and PTA-9055 are provided, wherein said progeny produces onion bulbs having the same (or better) low pungency, high soluble solid phenotypes and/or storage properties (at harvest and/or after storage) as described herein and/or as the deposited lines PTA-9053, PTA-9054 or PTA-9055.

Derivatives also include plants obtained from tissue culture methods and tissue cultures themselves, whereby tissue of any of the herein described plants is used (e.g. leaf, pollen, flowers, embryos, protoplasts, etc.). Likewise, transgenic onions of any of the above plants are encompassed herein. Thus, onion plants into which one or more genetic elements have been introduced by transformation are also encompassed herein. Transformation and regeneration of onion uses methods known in the art. For example, one or more genes for herbicide resistance or resistance against microorganisms may be introduced. Likewise, transgenes may be introduced into the onions according to the invention by crossing the onion plant with a plant comprising the transgene(s) and selecting offspring comprising the transgene(s).

Preferably there is no significant increase in pungency and/or no significant reduction in SSC during the storage period of the bulbs, i.e., when comparing the average pungency and/or average SSC at harvest (or shortly after harvest) with the average pungency and/or average SSC level after 2, 3, 4, 5, 6, 7 or more months of storage. “No significant increase in pungency” refers herein to an increase in pungency measurement (i.e., pyruvate) after the storage period by less than 10%, preferably less than 5%, more preferably less than 3%, 2% or 1%, more preferably no increase at all, and optionally even a reduction in pungency, compared to the measurement at harvest (or shortly after harvest). A reduction in pungency compared to pungency at harvest includes, for example, a reduction by at least 0.5%, 1% or more. “No significant reduction in SSC” refers herein to a reduction in SSC levels after the storage period of less than 2%, preferably less than 1% or 0.5%, more preferably unchanged, compared to the SSC level at harvest (or shortly after harvest).

Also the pungency range preferably remains narrow during storage. In addition, decay after at least 2, 3, 4, 5, 6, 7 or more months of storage (as can be measured visually and/or by weight, e.g., weighing non-decayed bulbs and comparing their weight to the total weight at the beginning of storage), is low, i.e., at any given time-point, e.g., after about 2, 2.5, 3, 3.5, 4, 4.5, 5, 5.5 or 6 months of storage or more, decay is less than 10%, preferably less than 9%, 8%, 7.2%, 7%, 6%, 5%, 4%, 3%, 2%, 1% or even less.

The onion plants, bulbs and seeds are long-day onions, i.e., the plants initiate bulb formation under long periods of contiguous light, e.g. artificial or natural light of at least about 14 hours or more. The onion plant thus preferably requires 14 or more hours per day (per 24 hours) of contiguous light in order to initiate bulb formation.

In one aspect, the onion plants, or a herein are capable of forming bulbs which have a pungency of less than 3.75/g FW, preferably equal to or less than 3.5, 3.0, 2.5, 2.3, 2.0, 1.8, 1.5, 1.3 μMol/g FW when measured 2, 3, 4, 5, 6 or 7 months after harvest, e.g. after 5-6 months of storage in the dark, under cool temperatures and at a RH of 60-80%. These bulbs have a significantly lower average pungency than bulbs of seeds deposited under NCIMB Accession numbers 41329 and 41330 (described in WO2007/011857), as well as preferably a narrower pungency range than various onion lines described in WO2007/011857. In addition, the bulbs have at least an equivalent, preferably a significantly higher average SSC content than bulbs of seeds deposited under NCIMB Accession numbers 41329 and 41330 and/or a longer storage ability compared to such bulbs.

It was found that bulbs and plants having very low pungency and high SSC content can be selected for, which was believed to be impossible. Without limiting the invention, it is thought that the genetic linkage between one or more regions responsible for high pungency and regions responsible for high SSC can, contrary to prior belief, be broken, enabling the selection or identification of low pungency/high SSC plants. Plants provided herein can be made as described in the methods and Examples herein below, using breeding and selection methods (PAD measurements, SSC measurements and/or storage decay measurements and the like). Also seeds provided herein can be used to make plants according to the invention, as the traits can be transferred from the deposited seeds to other onion plants by crossing and selection. Basically the traits (low pungency, high SSC and/or long storage ability) can be introduced into any long day onion, such as for example Spanish onions, Spanish-type onions, (northern) yellow-type onions, white and red type onions, hard-globe eastern or western type onions, etc. Onion plants (e.g., open pollinated or hybrid plants) can therefore be made having these traits and having good agronomic characteristics, such as disease resistance (e.g. Fusarium resistance), pink root resistance, bulb size, % single centers, bolting tolerance, etc.

Also provided herein are containers comprising a plurality of onion bulbs having the above phenotypes, as well as containers comprising a plurality of onion seeds of the above plants or containers comprising a plurality of onion plants or seedlings. Containers may be of any type, such as bags, cans, tins, trays, boxes, flats and the like. Also provided herein are containers comprising onion bulbs having an average PAD measurement of less than about 5.5 μMol/g FW, preferably less than 3.75, 3.5, 3.0, etc. μMol/g FW, high SSC and/or long storage ability (each phenotype as defined above). Preferably in a container at least 75%, 85%, 95%, 98% or more of the bulbs have such a PAD measurement, SSC level and/or storage ability. Also, preferably the pungency range of all bulbs in a container is narrow. A container preferably contains at least about 1 pound, 5 pounds, 10 pounds or more bulbs. The container may be in any location, e.g., a store (such as a grocery store), warehouse, market place, distributor, etc.

Seed containers comprising seeds of an onion plant requiring 14 or more hours contiguous light to initiate bulb formation, wherein the seed is capable of producing an onion plant having a bulb comprising low pungency (i.e., a PAD measurement as defined), high SSC and/or long storage ability (also as defined) are also provided. Preferably the onion bulbs from greater than 50%, more preferably from greater than 60%, 70%, 75%, 80%, 90%, 95%, 98% of the plants produced by such seeds produce bulbs having an average PAD measurement of less than 5.5 μMol/g FW, preferably less than 5.0, 4.0, 3.5, or 3.0 μMol/g FW, etc., high SSC and/or long storage ability. The containers preferably comprise at least 100, 500, 1000, 10.000 or more seeds and is preferably selected from a bag, box, packet, tin or can. Also, preferably the pungency range of all bulbs derivable from the seeds in a container is narrow.

In another aspect, a method for producing an onion plant or seed, or a group of plants or seeds, is provided, whereby the plant, or group of plants, produce(s) a bulb after exposure to at least about 14 hours light per day (during a period of at least about 1 or more weeks, e.g., 2 or 3 or more weeks) which comprises a (single bulb or mean) pungency of less than 5.5, 5.0, 4.0, 3.75 μMol/g FW at harvest (or less, as defined above) and a (single bulb or mean) SSC at harvest of at least 7.5% or more (as defined above). Preferably, the bulbs retain low pungency and high SSC during storage. Also, the pungency range of the bulbs is preferably narrow. The method comprises crossing two parent onion plants or selfing an onion plant and harvesting the resulting onion seeds from the cross or selfing, wherein at least one parent is an onion plant as described above, or a derivative thereof. Seeds produced by the method are also provided herein, as are onion plants produced by growing those seeds and onion bulbs harvested from those grown plants.

The method may further comprise the step of growing an F1 hybrid onion plant obtained from seed obtained from said cross, crossing the F1 onion plant to another onion plant, e.g., to one of the parents used, and selecting progeny onion plants having the desired low pungency and high SSC content.

In a further aspect, a method for producing an onion plant or seed or a group of onion plants or seeds, is provided, whereby the plant, or group of plants, produce(s) (a) bulb(s) after exposure to at least about 14 hours light per day (during a period of at least about 1 or more weeks, e.g., 2 or 3 or more weeks) which comprises a (single bulb or mean) pungency of less than 5.5, 5.0, 4.0, 3.75 μMol/g FW at harvest (or less, as defined above) and a (single bulb or mean) SSC at harvest of at least 7.0 or 7.5% or more (as defined above). Preferably, the bulbs retain low pungency and high SSC during storage, show little decay during storage and/or have a narrow pungency range (all as described). The method comprises the steps of: a) crossing an onion plant producing bulbs having a low SSC and low pungency with an onion plant producing bulbs having a high SSC and high pungency, b) obtaining the F1 seeds from said cross, c) selling and/or crossing the plants obtained from the F1 seeds one or more times with one another or with other onion plants, and d) identifying and selecting progeny plants which produce bulbs having a low pungency and high SSC by phenotyping the bulbs.

Optionally steps c) and/or d) can be repeated several times. Crossing in step c) may also involve backcrossing. In step d), plants having a narrow pungency range and/or plants showing little decay during storage may be selected. Thus, pungency range and/or storage ability can also be used as selection criteria in addition to or as an alternative of low pungency and/or high SSC. The same applies to the methods described herein below, even if only SSC and pungency are mentioned.

The phenotyping preferably involves determining the pungency, SSC content and/or storage ability (e.g. percentage decay after a certain storage period, which can be analysed visually) of the bulbs (e.g. by phenotyping one or more populations of step c) above) and selecting rare recombinants or mutants which have a low pungency and/or high SSC and/or long storage ability. The plants used under a) may be commercially available onion cultivars or breeding lines, such as long day onions and short day onions. Phenotyping can be carried out on a plurality of single bulbs independently, preferably grown under the same conditions next to suitable controls, or on a sample composed of (all or parts of) several bulbs. When single bulbs are used, preferably the mean value is calculated from a representative number of bulbs. Phenotyping can be done one or more times. For example PAD measurements and/or SSC measurements may be carried out at harvest and after 1, 2, 3, 4 or more weeks of storage or 2, 3, 4, 5, 6, 7, 8, 9 or more months of storage. In one embodiment the phenotyping (PAD and/or SSC measurements) is carried out after about 5, 6 or 7 months of storage (e.g. after about 150-210 days, e.g. about 150 days, 180 days, 200 days or 205, 206, 207, 208, 209, 210 days of storage). Phenotyping can be carried out at one or more steps of a breeding scheme.

Phenotyping may also comprise an analysis of the photoperiod response and selection of plants having a long-day response, so that in step d) long day onions are produced.

In one aspect a method for making long-day onion plants comprising a low pungency and high SSC, is provided, comprising a) (optionally) analyzing onion bulbs for pungency and SSC, b) crossing plants producing bulbs having a high pungency and high SSC with plants producing bulbs with a low pungency and low SSC to produce F1 hybrids, c) selfing and/or (back)crossing F1 hybrid plants one or more times and d) selecting progeny plants for low pungency and high SSC content (at harvest and/or after storage) and preferably also for having a long day length photoperiod response and/or preferably also for having a narrow pungency range and e) selecting a long day onion plant producing bulbs having low pungency and high SSC, with levels similar to those of lines PTA-9053, PTA-9054 or PTA-9055 at harvest and/or after storage. Step d) involves pungency and SSC analysis at harvest and/or after storage. In the initial cross, the low pungency, low SSC onion parent may be a short-day onion variety, cultivar or breeding line and the high pungency, high SSC may be a long day onion variety, cultivar or breeding line. Preferably steps c) and d) are repeated several times, so that several cycles of phenotypic recurrent selection are carried out, leading to long day onions of step e).

In yet a further aspect, a method of producing an inbred, long-day onion plant comprising low pungency and high SSC is provided herein, comprising the steps of: a) the creation of variable populations of Allium cepa comprising the steps of crossing a plant or plants producing bulbs with low pungency and high SSC (as described herein) with a plant of the species Allium cepa, b) harvesting the F1 seed from any of the plants used in the cross of a) and growing F1 plants from the seed harvested, c) selfing the plants grown under b) or crossing these plants amongst one another, or crossing these plants with plants of Allium cepa, d) growing plants from the resulting seed harvested under normal plant growing conditions and, e) selecting plants producing bulbs having low pungency and high SSC, followed by selfing the selected plants, and optionally f) repeating the steps d) and/or e) until the inbred lines are obtained which are homozygous and can be used as parents in the production of hybrids having low pungency and high SSC.

Also provided is a method for developing male sterile inbred lines with the properties of low pungency and high SSC comprising the steps of crossing the plants of the inbred lines described above with plants of male sterile lines of Allium cepa and the subsequent selection and recurrent back crossing with the male fertile parent until the new male sterile line is genetically and phenotypically similar to the male fertile recurrent parent inbred line and has the combination of low pungency and high SSC.

The male sterile inbred line may be crossed with a male fertile inbred line resulting in hybrid seeds, whereby the plants grown therefrom possess the properties of low pungency and high SSC.

Likewise open pollinated, long-day onion plants comprising low pungency and high SSC can be made. Thus, onion plants according to the invention may be maintained as open pollinated lines, half-sib lines, male sterile lines, female sterile lines, etc. Male sterile inbred lines of onion plants according to the invention are useful as parents for producing hybrids.

In another aspect, a method for producing an onion crop from onion seeds or plants according to the invention and long day onions harvested therefrom is provided.

In another aspect, the invention provides an Allium cepa plant, or plant part or seed thereof, comprising in its genome one or more of QTL1 on chromosome 1, QTL2 on chromosome 2 and/or QTL7 on chromosome 7, wherein said QTL1, QTL2 and QTL7 confer a reduced pyruvate level,

wherein QTL1 is comprised in an introgression fragment comprising a haplotype of one or more of the following SNP markers:
SNP_16 comprising an Adenine at nucleotide 51 of SEQ ID NO: 31 or at nucleotide 51 of a sequence comprising at least 97% identity to SEQ ID NO: 31;
SNP_17 comprising an Adenine at nucleotide 51 of SEQ ID NO: 33 or at nucleotide 51 of a sequence comprising at least 97% identity to SEQ ID NO: 33;
SNP_05 comprising a Cytosine at nucleotide 51 of SEQ ID NO: 9 or at nucleotide 51 of a sequence comprising at least 97% identity to SEQ ID NO: 9;
SNP_06 comprising an Adenine at nucleotide 51 of SEQ ID NO: 11 or at nucleotide 51 of a sequence comprising at least 97% identity to SEQ ID NO: 11;
SNP_07 comprising a Cytosine at nucleotide 51 of SEQ ID NO: 13 or at nucleotide 51 of a sequence comprising at least 97% identity to SEQ ID NO: 13;
SNP_08 comprising a Thymine at nucleotide 51 of SEQ ID NO: 15 or at nucleotide 51 of a sequence comprising at least 97% identity to SEQ ID NO: 15;
SNP_18 comprising a Thymine at nucleotide 51 of SEQ ID NO: 35 or at nucleotide 51 of a sequence comprising at least 97% identity to SEQ ID NO: 35;
SNP_19 comprising a Thymine at nucleotide 51 of SEQ ID NO: 37 or at nucleotide 51 of a sequence comprising at least 97% identity to SEQ ID NO: 37;
SNP_20 comprising a Guanine at nucleotide 51 of SEQ ID NO: 39 or at nucleotide 51 of a sequence comprising at least 97% identity to SEQ ID NO: 39; and/or
SNP_21 comprising a Cytosine at nucleotide 51 of SEQ ID NO: 41 or at nucleotide 51 of a sequence comprising at least 97% identity to SEQ ID NO: 41,

wherein QTL2 is comprised in an introgression fragment comprising a haplotype of one or more of the following SNP markers:
SNP_11 comprising an Adenine at nucleotide 51 of SEQ ID NO: 21 or at nucleotide 51 of a sequence comprising at least 97% identity to SEQ ID NO: 21;
SNP_12 comprising a Cytosine at nucleotide 51 of SEQ ID NO: 23 or at nucleotide 51 of a sequence comprising at least 97% identity to SEQ ID NO: 23;
SNP_13 comprising a Thymine at nucleotide 51 of SEQ ID NO: 25 or at nucleotide 51 of a sequence comprising at least 97% identity to SEQ ID NO: 25;
SNP_14 comprising an Adenine at nucleotide 51 of SEQ ID NO: 27 or at nucleotide 51 of a sequence comprising at least 97% identity to SEQ ID NO: 27;
SNP_01 comprising a Thymine at nucleotide 51 of SEQ ID NO: 1 or at nucleotide 51 of a sequence comprising at least 97% identity to SEQ ID NO: 1;
SNP_02 comprising an Adenine at nucleotide 51 of SEQ ID NO: 3 or at nucleotide 51 of a sequence comprising at least 97% identity to SEQ ID NO: 3;
SNP_03 comprising a Cytosine at nucleotide 51 of SEQ ID NO: 5 or at nucleotide 51 of a sequence comprising at least 97% identity to SEQ ID NO: 5;
SNP_04 comprising a Thymine at nucleotide 51 of SEQ ID NO: 7 or at nucleotide 51 of a sequence comprising at least 97% identity to SEQ ID NO: 7; and/or
SNP_15 comprising a Guanine at nucleotide 51 of SEQ ID NO: 29 or at nucleotide 51 of a sequence comprising at least 97% identity to SEQ ID NO: 29, and/or

wherein QTL7 is comprised in an introgression fragment comprising a haplotype of one or more of the following SNP markers:
SNP_22 comprising a Thymine at nucleotide 51 of SEQ ID NO: 43 or at nucleotide 51 of a sequence comprising at least 97% identity to SEQ ID NO: 43;
SNP_23 comprising a Thymine at nucleotide 51 of SEQ ID NO: 45 or at nucleotide 51 of a sequence comprising at least 97% identity to SEQ ID NO: 45;
SNP_24 comprising a Cytosine at nucleotide 51 of SEQ ID NO: 47 or at nucleotide 51 of a sequence comprising at least 97% identity to SEQ ID NO: 47;
SNP_25 comprising a Guanine at nucleotide 51 of SEQ ID NO: 49 or at nucleotide 51 of a sequence comprising at least 97% identity to SEQ ID NO: 49;
SNP_09 comprising a Thymine at nucleotide 51 of SEQ ID NO: 17 or at nucleotide 51 of a sequence comprising at least 97% identity to SEQ ID NO: 17; and/or
SNP_10 comprising a Guanine at nucleotide 51 of SEQ ID NO: 19 or at nucleotide 51 of a sequence comprising at least 97% identity to SEQ ID NO: 19.

In another aspect, the plant comprises one, two, or three of QTL1 on chromosome 1, QTL2 on chromosome 2 and/or QTL7 on chromosome 7. In other aspects, QTL1 is comprised in an introgression fragment comprising a haplotype of two, three, four, five, or more of the following SNP markers: SNP_16, SNP_17, SNP_05, SNP_06, SNP_07, SNP_08, SNP_18, SNP_19, SNP_20 and/or SNP_21. In other aspects, QTL2 is comprised in an introgression fragment comprising a haplotype of two, three, four, five or more of the following SNP markers: SNP_11, SNP_12, SNP_13, SNP_14, SNP_01, SNP_02, SNP_03, SNP_04 and/or SNP_15. In other aspects, QTL7 is comprised in an introgression fragment comprising a haplotype of two, three, four, five, or more of the following SNP markers: SNP_22, SNP_23, SNP_24, SNP_25, SNP_09 and/or SNP_10. In other aspects, the plant comprises any combination of SNP markers SNP_1 to SNP_25, such as SNP_3, SNP_7, and SNP_10. In other aspects, the plant is a single cross F1 hybrid or an inbred line.

In another aspect, the invention provides for a method of producing an Allium cepa plant comprising in its genome one or more of QTL1 on chromosome 1, QTL2 on chromosome 2 and/or QTL7 on chromosome 7, wherein said QTL1, QTL2 and QTL7 confer a reduced pyruvate level, said method comprising: a) crossing a first onion plant comprising in its genome one or more of QTL1, QTL2 and/or QTL7 with a second onion plant, and b) collecting seeds from said cross,

wherein QTL1 is comprised in an introgression fragment comprising a haplotype of one or more of the following SNP markers:
SNP_16 comprising an Adenine at nucleotide 51 of SEQ ID NO: 31 or at nucleotide 51 of a sequence comprising at least 97% identity to SEQ ID NO: 31;
SNP_17 comprising an Adenine at nucleotide 51 of SEQ ID NO: 33 or at nucleotide 51 of a sequence comprising at least 97% identity to SEQ ID NO: 33;
SNP_05 comprising a Cytosine at nucleotide 51 of SEQ ID NO: 9 or at nucleotide 51 of a sequence comprising at least 97% identity to SEQ ID NO: 9;
SNP_06 comprising an Adenine at nucleotide 51 of SEQ ID NO: 11 or at nucleotide 51 of a sequence comprising at least 97% identity to SEQ ID NO: 11;
SNP_07 comprising a Cytosine at nucleotide 51 of SEQ ID NO: 13 or at nucleotide 51 of a sequence comprising at least 97% identity to SEQ ID NO: 13;
SNP_08 comprising a Thymine at nucleotide 51 of SEQ ID NO: 15 or at nucleotide 51 of a sequence comprising at least 97% identity to SEQ ID NO: 15;
SNP_18 comprising a Thymine at nucleotide 51 of SEQ ID NO: 35 or at nucleotide 51 of a sequence comprising at least 97% identity to SEQ ID NO: 35;
SNP_19 comprising a Thymine at nucleotide 51 of SEQ ID NO: 37 or at nucleotide 51 of a sequence comprising at least 97% identity to SEQ ID NO: 37;
SNP_20 comprising a Guanine at nucleotide 51 of SEQ ID NO: 39 or at nucleotide 51 of a sequence comprising at least 97% identity to SEQ ID NO: 39; and/or
SNP_21 comprising a Cytosine at nucleotide 51 of SEQ ID NO: 41 or at nucleotide 51 of a sequence comprising at least 97% identity to SEQ ID NO: 41,

wherein QTL2 is comprised in an introgression fragment comprising a haplotype of one or more of the following SNP markers:
SNP_11 comprising an Adenine at nucleotide 51 of SEQ ID NO: 21 or at nucleotide 51 of a sequence comprising at least 97% identity to SEQ ID NO: 21;
SNP_12 comprising a Cytosine at nucleotide 51 of SEQ ID NO: 23 or at nucleotide 51 of a sequence comprising at least 97% identity to SEQ ID NO: 23;
SNP_13 comprising a Thymine at nucleotide 51 of SEQ ID NO: 25 or at nucleotide 51 of a sequence comprising at least 97% identity to SEQ ID NO: 25;
SNP_14 comprising an Adenine at nucleotide 51 of SEQ ID NO: 27 or at nucleotide 51 of a sequence comprising at least 97% identity to SEQ ID NO: 27;
SNP_01 comprising a Thymine at nucleotide 51 of SEQ ID NO: 1 or at nucleotide 51 of a sequence comprising at least 97% identity to SEQ ID NO: 1;
SNP_02 comprising an Adenine at nucleotide 51 of SEQ ID NO: 3 or at nucleotide 51 of a sequence comprising at least 97% identity to SEQ ID NO: 3;
SNP_03 comprising a Cytosine at nucleotide 51 of SEQ ID NO: 5 or at nucleotide 51 of a sequence comprising at least 97% identity to SEQ ID NO: 5;
SNP_04 comprising a Thymine at nucleotide 51 of SEQ ID NO: 7 or at nucleotide 51 of a sequence comprising at least 97% identity to SEQ ID NO: 7; and/or
SNP_15 comprising a Guanine at nucleotide 51 of SEQ ID NO: 29 or at nucleotide 51 of a sequence comprising at least 97% identity to SEQ ID NO: 29, and/or

wherein QTL7 is comprised in an introgression fragment comprising a haplotype of one or more of the following SNP markers:
SNP_22 comprising a Thymine at nucleotide 51 of SEQ ID NO: 43 or at nucleotide 51 of a sequence comprising at least 97% identity to SEQ ID NO: 43;
SNP_23 comprising a Thymine at nucleotide 51 of SEQ ID NO: 45 or at nucleotide 51 of a sequence comprising at least 97% identity to SEQ ID NO: 45;
SNP_24 comprising a Cytosine at nucleotide 51 of SEQ ID NO: 47 or at nucleotide 51 of a sequence comprising at least 97% identity to SEQ ID NO: 47;
SNP_25 comprising a Guanine at nucleotide 51 of SEQ ID NO: 49 or at nucleotide 51 of a sequence comprising at least 97% identity to SEQ ID NO: 49;
SNP_09 comprising a Thymine at nucleotide 51 of SEQ ID NO: 17 or at nucleotide 51 of a sequence comprising at least 97% identity to SEQ ID NO: 17; and/or
SNP_10 comprising a Guanine at nucleotide 51 of SEQ ID NO: 19 or at nucleotide 51 of a sequence comprising at least 97% identity to SEQ ID NO: 19.

In another aspect of the above-mentioned method, the first onion plant comprises one, two or three of QTL1 on chromosome 1, QTL2 on chromosome 2 and/or QTL7 on chromosome 7. In other aspects, QTL1 is comprised in an introgression fragment comprising a haplotype of two, three, four, five, or more of the following SNP markers: SNP_16, SNP_17, SNP_05, SNP_06, SNP_07, SNP_08, SNP_18, SNP_19, SNP_20 and/or SNP_21. In other aspects, QTL2 is comprised in an introgression fragment comprising a haplotype of two, three, four, five or more of the following SNP markers: SNP_11, SNP_12, SNP_13, SNP_14, SNP_01, SNP_02, SNP_03, SNP_04 and/or SNP_15. In other aspects, QTL7 is comprised in an introgression fragment comprising a haplotype of two, three, four, five, or more of the following SNP markers: SNP_22, SNP_23, SNP_24, SNP_25, SNP_09 and/or SNP_10. In other aspects, the plant comprises any combination of SNP markers SNP_1 to SNP_25, such as SNP_3, SNP_7, and SNP_10. In other aspects, the produced Allium cepa plant is a single cross F1 hybrid or an inbred line.

The following non-limiting examples illustrate the production of onion plants, seeds and bulbs according to the invention. All references mentioned herein are incorporated by reference.

### EXAMPLES

### Example 1—Plant Development

Plants have been obtained by a long term breeding program (Oregon, USA) in which numerous plants have been analyzed for the desired combination of the traits as mentioned.

The initial cross concerned low pungency/low SSC onion plants (the commercially available long-day variety Ailsa Craig) with high pungency/high SSC long-day onion plants of a breeding line designated I37787B and subsequent selfing of the F1 plants to create variable F2 populations. No Spanish background was used.

A selected F2 individual (phenotyped for pungency and SSC) was crossed to an individual selected from a breeding line derived from the variety Oregon Danvers Yellow Globe. Plants from this cross where selfed and selected individuals from this progeny were selfed again. A low pungency, high SSC line was obtained and designated 137554.

Six additional cycles of phenotypic recurrent selection were made, with selection for the phenotypes high SSC and low pungency and having all desirable agronomic traits and long day length photoperiod response. High SSC was determined using refractometry analysis according to the method of Mann and Hoyle, 1945 (Proc. Americ. Soc. Hort. Sci. 46: 285-292) or Foskett and Peterson, 1949 (Proc. Americ. Soc. Hort. Sci. 55: 314-318). Pungency was determined using the PAD measurement of Schwimmer and Weston 1961 {supra). Throughout the scheme plants in each generation were selected after approximately 150 days of storage, i.e., PAD and/or SSC measurements were made after about 5 to 6 months of storage for selection purposes.

Coincident with the six cycles of phenotypic recurrent selection the selected plants were crossed and backcrossed to cytoplasmic/nuclear male sterile plants. In this way inbred maintainer line I37554B and its male sterile companion line I37554A were developed, both of which have lower pungency and higher SSC at harvest and after storage and are long day onions. Seeds of 137554 (I37554A and I37554B) have been deposited at the ATCC under the Budapest Treaty under accession number PTA-9054 and PTA-9055, respectively.

Line I37853B was developed by further breeding and selection with the above material and seeds of I37853B were deposited at the ATCC under the Budapest Treaty under accession number PTA-9053. Line I37853B has even lower pungency than I37554A and B and has improved bulb quality.

From these plants parent lines for producing hybrid varieties have been developed by additional crossing and further inbreeding while selecting for agronomic traits and good combining ability. The hybrid varieties produced with these lines have been evaluated for the unique combination of low pungency/high soluble solids, long storage and other desirable agronomic characteristics.

In one aspect of the invention, novel plants, seeds and bulbs of long-day onion I37554A or B and of I37853B are provided. Also, hybrids produced from crossing I37554A or B and I37853B are provided, as well as plants produced from such crosses or selfings and which produce bulbs comprising low pungency, high SSC and/or high storage capabilities.

### Example 2—Plant 137554 Having Low Pungency and High SSC

Table 1 below shows (average) pungency measured as pyruvate concentration in μMol/g FW and SSC content (%) of bulbs of the plant designated I37554A and the commercially available Long Day variety Granero 9536, both at harvest and during 3 months of storage.

TABLE 1

| | |
|---|---|
| Pyruvate (μMol/g FW) | SSC (%) |

| Time Period* | Granero | I37554 | Granero | I37554 |
|---|---|---|---|---|
| 1 | 6.22 | 4.92 | 8.6 | 8.3 |
| 2 | 5.95 | 5.34 | 9.3 | 8.5 |
| 3 | 5.91 | 5.5 | 9.7 | 8.9 |
| 4 | 6.92 | 6.17 | 8.3 | 8.1 |
| 5 | 8.66 | 5.78 | 9.4 | 8.1 |
| 6 | 9.07 | 3.33 | 8.4 | 8.2 |
| 7 | 8.53 | 4.89 | 8.1 | 7.5 |
| 8 | 9.97 | 4.62 | 8.5 | 7.4 |

*The time periods are approximately two weeks apart.

The Example shows that during storage pyruvate levels of Granero, a long day Spanish hybrid variety which is pungent and has high SSC, increases significantly, while the pungency of I37554A does not change significantly and the SSC levels remain high and constant. Also, at harvest I37554A, combines low pungency with high SSC (and long day characteristics).

### Example 3

Table 2 shows yield and percent storage decay of I37554A compared to commercial pungent and high SSC varieties Granero and Nebula (136 days after harvest, i.e., after about 4.5 months of storage). Percent decay was assessed by weight.

TABLE 2

| | | | | | | Storage decay | |
|---|---|---|---|---|---|---|---|
| | | | | | Weight | | No |
| | Yield (lbs/acre - 100's) | | | | Weight/bulb | Decay | decay |
| Plant | Total | Large | Medium | Small | (g) | (%) | (%) |
| I37554A | 595 | 401 | 134 | 14.5 | 179.5 | 7.1 | 92.9 |
| Granero | 680 | 539 | 104 | 3.5 | 225.0 | 5.5 | 94.5 |
| Nebula | 380 | 169 | 182 | 11.4 | 142.7 | 3.4 | 96.6 |

The Example shows that, while having low pungency, line I37554 A has similar, good storage characteristics as known pungent storage onions which have high SSC.

### Example 4

Table 3 shows pungency and SSC data for I37554B after more than 6 months (207 days, i.e., 6.9 months) of storage. Table 3 shows data for 92 single bulbs of I37554B and the mean.

TABLE 3

| | Pungency | |
|---|---|---|
| Plot number | (μMol/g FW) | SSC (%) |
| 6111 | 2.52 | 9.20 |
| 6111 | 4.15 | 10.20 |
| 6111 | 2.70 | 9.20 |
| 6111 | 3.63 | 9.20 |
| 6111 | 2.62 | 9.80 |
| 6111 | 3.22 | 8.80 |
| 6111 | 4.37 | 8.80 |
| 6111 | 2.41 | 10.20 |
| 6111 | 3.56 | 10.20 |
| 6111 | 3.57 | 8.20 |
| 6111 | 3.03 | 7.80 |
| 6111 | 4.50 | 10.20 |
| 6111 | 3.88 | 10.20 |
| 6111 | 2.77 | 7.60 |
| 6111 | 3.48 | 9.20 |
| 6111 | 3.42 | 9.00 |
| 6111 | 3.54 | 10.40 |
| 6111 | 2.82 | 9.20 |
| 6111 | 4.41 | 10.80 |
| 6111 | 1.90 | 9.40 |
| 6111 | 2.86 | 9.80 |
| 6111 | 2.67 | 9.40 |
| 6111 | 3.29 | 9.80 |
| 6111 | 2.69 | 8.80 |
| 6111 | 4.03 | 9.00 |
| 6111 | 2.86 | 8.80 |
| 6111 | 2.76 | 8.80 |
| 6111 | 3.04 | 8.40 |
| 6111 | 3.50 | 9.40 |
| 6111 | 4.29 | 9.40 |
| 6111 | 4.13 | 7.80 |
| 6111 | 3.25 | 8.60 |
| 6111 | 2.05 | 9.80 |
| 6111 | 3.93 | 10.20 |
| 6111 | 3.42 | 9.80 |
| 6111 | 3.56 | 10.40 |
| 6111 | 3.15 | 8.20 |
| 6111 | 3.59 | 10.20 |
| 6111 | 2.86 | 10.20 |
| 6111 | 3.16 | 10.20 |
| 6111 | 2.36 | 8.60 |
| 6111 | 4.15 | 10.80 |
| 6111 | 3.20 | 9.00 |
| 6111 | 2.49 | 9.40 |
| 6111 | 3.35 | 10.80 |
| 6111 | 4.47 | 10.60 |
| 6111 | 3.87 | 9.80 |
| 6111 | 3.87 | 9.60 |
| 6111 | 4.17 | 10.40 |
| 6111 | 2.59 | 9.80 |
| 6111 | 1.29 | 10.20 |
| 6111 | 4.08 | 9.60 |
| 6111 | 3.25 | 8.80 |
| 6111 | 2.43 | 8.60 |
| 6111 | 3.10 | 9.40 |
| 6111 | 4.54 | 8.20 |
| 6111 | 4.12 | 9.80 |
| 6111 | 3.57 | 10.40 |
| 6111 | 3.62 | 10.40 |
| 6111 | 3.09 | 8.40 |
| 6111 | 2.92 | 9.00 |
| 6111 | 4.44 | 10.20 |
| 6111 | 1.76 | 9.80 |
| 6111 | 4.39 | 9.80 |
| 6111 | 3.23 | 10.40 |
| 6111 | 3.69 | 10.20 |
| 6111 | 4.20 | 11.20 |
| 6111 | 4.35 | 9.40 |
| 6111 | 4.29 | 10.40 |
| 6111 | 4.14 | 10.20 |
| 6111 | 4.43 | 10.20 |
| 6111 | 3.97 | 10.20 |
| 6111 | 3.72 | 10.40 |
| 6111 | 3.53 | 10.40 |
| 6111 | 4.17 | 11.60 |
| 6111 | 3.67 | 10.20 |
| 6111 | 4.30 | 10.00 |
| 6111 | 3.41 | 9.40 |
| 6111 | 4.17 | 9.80 |
| 6111 | 2.85 | 9.00 |
| 6111 | 4.74 | 9.80 |
| 6111 | 3.14 | 9.80 |
| 6111 | 4.17 | 10.00 |
| 6111 | 3.80 | 11.20 |
| 6111 | 3.83 | 10.20 |
| 6111 | 3.75 | 10.20 |
| 6111 | 2.84 | 9.80 |
| 6111 | 4.32 | 9.40 |
| 6111 | 4.63 | 10.40 |
| 6111 | 3.04 | 9.60 |
| 6111 | 3.78 | 10.00 |
| 6111 | 4.84 | 9.80 |
| MEAN | 3.5 | 9.67 |

The data show that line I37554B has low pungency and high SSC levels. Even after more than 6 months of storage the average pungency remains very low and average SSC remains high. Also, the pungency range is narrow (min. 1.29, max. 4.85).

### Example 5—Line I37853B

Table 4 shows single bulb pungency levels of line I37853B after 5-6 months of storage showing that very low pungency (mean pungency 2.3 μMol/g FW) has been achieved, combined with high SSC.

TABLE 4
Pungency (μMol/g FW)
1.3
1.4
3.7
4.7
3.3
2.3
3.1
2.1
3.0
2.0
2.6
2.1
2.4
1.6
2.4
2.3
2.2
3.0
1.2
2.6
1.9
2.1
3.6
2.0
1.8
2.0
3.0
1.3
2.1
1.8
1.3
2.4
2.2
2.5
1.9
2.0
3.7
1.9
Mean (38 bulbs) = 2.3

### Example 6—Hybrids

To generate hybrids of long day storage onions having low pungency and high SSC at harvest and after long term storage, line I37554A (female parent) was crossed with I37853B (male parent) to generate F1 hybrid seeds. The hybrids will be grown at various locations and pyruvate and SSC levels will be assessed and compared to the parents and high pungency lines or cultivars.

### Example 7—Line I37853B

Table 5 shows average pungency (pyruvate FW), SSC (%) and Storage Decay (%) of line 137554B after storage (132 days after harvest).

TABLE 5

| | Pyruvate | | Storage Decay | |
|---|---|---|---|---|
| Name | (μMol/g FW) | SSC (%) | (%) | No decay (%) |
| I375548 | 3.8 | 8.6 | 9.1 | 90.9 |

### Example 8—QTL Mapping

A single low pungency (reduced pungency) line 137720B, derived from a cross between 137554B and material in the pedigree of I37554B, was crossed with two pungent inbred lines I37977B (“population 1”) and 137545-7 (“population 2”). The resulting F1 hybrids were self-pollinated to produce two segregating populations of F2 plants. In population 1, a total of 331 F2 plants were grown, and in population 2, a total of 236 plants were grown under field conditions in Brooks, Oreg. Leaf tissue was collected from each F2 individual for DNA extraction. Bulbs were harvested and stored for four months, after which the pyruvate level was measured.

A panel of 283 KASP markers was run on the DNA extracted from each F2 individual. Monomorphic markers were discarded, and a genetic linkage map was calculated for each population independently using Kosambi's mapping function in the JoinMap software package. The resulting maps were used in combination with the pyruvate measurements to identify quantitative trait loci (QTL) using the interval mapping method in the MapQTL software package. The logarithm of the odds (LOD) score threshold for significant association of loci with pyruvate concentration was set using the genome wide permutation testing method with a cumulative count of at least 0.95, with 200 iterations of 1,000 permutations for each population.

In population 1, there were two significant QTL detected which exceeded the calculated LOD threshold, one on linkage group 3 and one on linkage group 6. Population 2 contained a single significant QTL on linkage group 4. Peak markers for the three QTL were identified on an integrated genetic linkage map and additional nearby markers were added to the F2 population map. The number of additional markers added is detailed in Table 1. Information on the peak and flanking markers for each significant QTL is in Table 2.

After integrating additional markers, the peak marker for the QTL on linkage group 3 explained 5.6% of the variation in pyruvic acid values, the peak marker for the QTL on linkage group 4 explained 45.6% of the variation, and the peak marker for the QTL on linkage group 6 explained 6.5% of the variation in pyruvic acid values.

TABLE 6
Additional markers added at each of the three significant QTL

| | Additional | | | In QTL |
|---|---|---|---|---|
| QTL | markers | Monomorphic | Polymorphic | interval |
| Linkage group 3 | 19 | 8 | 11 | 1 |
| Linkage group 4 | 5 | 4 | 1 | 1 |
| Linkage group 6 | 12 | 5 | 7 | 4 |

TABLE 7
Names of peak and flanking markers
for each of the significant QTL

| QTL | Flanking | Peak | Flanking |
|---|---|---|---|
| Linkage group 3 | SNP_11 | SNP_03 | SNP_04 |
| Linkage group 4 | SNP_16 | SNP_07 | SNP_20 |
| Linkage group 6 | SNP_22 | SNP_10 | SNP_10* |

*SNP_10 is the most distal marker on linkage group 6, so there is no true flanking marker

TABLE 8
Nucleotide sequences of the identified
peak and flanking markers

| | |
|---|---|
| SNP_ | CATGGCAAGAAAAATGTTCCAAGCTTCTTGAAACATCTCCAG |
| 01 | ACAATTGG[T/G]CTTTCAAAAGTGATTCCGAAACCTCTACT |
| | TCGAAGTACATTGCTTCTTTA (SEQ ID NO: 1/2) |
| SNP_ | TTGATAAAAGCGATATTGTTGGGGAAGTATCCTGCAGAATTT |
| 02 | TTGTGGCC[G/A]TGAAAAGGAAGATGAGTGGAAGCTAAATG |
| | CATTGGAAATTGTGTTTGGCC (SEQ ID NO: 3/4) |
| SNP_ | GACGGATAATTCAGAACAGGGAGGAAGTAAGCAGCATGTGAT |
| 03 | AATCAACA[T/C]AGAGGATAGAGCTTCTGAGGTGGGTAAAG |
| | CTGATGCGAATCGTCCCACGC (SEQ ID NO: 5/6) |
| SNP_ | TGATGCTATCTGGAAGATTACAAATTTATAGATGGTATGGGA |
| 04 | TGTTGGAA[T/C]AGCAGATTGGTGCTGAATTTAGTACTACC |
| | AAAGGACAGATGACGCCGCTT (SEQ ID NO: 7/8) |
| SNP_ | CTCAGGAATGAGATTTGCATCAGCATCCTCAGGATCAATTTC |
| 05 | TTGATCAT[T/C]AGATTGCATTCCATAATTAAACAGCAGCT |
| | CGTGCCACTTCATGTCATCAA (SEQ ID NO: 9/10) |
| SNP_ | CCTGAGCGATGTAAAAGGAAGAGATAGAGTATGGGAGTTGAG |
| 06 | AAACTGTA[A/G]CCATGTTTTTCATAAAGGATGCCTGGACA |
| | AATGGTTAGAGCATGATGAGC (SEQ ID NO: 11/12) |
| SNP_ | GATTGTTTAGACATTCGTTGTATTTCGCGAGATCTGCTCACG |
| 07 | GGATAGCT[G/C]ATTTTTTAAGATTTTTCGAGAAATTCTAC |
| | CTGGATTTTTGTTAGGGTTTT (SEQ ID NO: 13/14) |
| SNP_ | AATTACACTTTAGCAATCAAGAGATGATTCTCAGGAGAAATA |
| 08 | TCCGAGGA[T/G]GTATACTTCACCATTTGTGCATCCAACCC |
| | CTGATCCCTTAGCCACGACAT (SEQ ID NO: 15/16) |
| SNP_ | GATGATGGTGGAGCGAGAAGAGAATGGTTCTGGTTTGTGGTT |
| 09 | TGATTGGA[T/C]GGGTTTGTCGATCGAGGTCCATGACCATT |
| | GCTTGTGGATGCGGTTCCATC (SEQ ID NO: 17/18) |
| SNP_ | GAGCAAGATCAGTTAAGATTCTTAAAGCTCTTTGAAGACACC |
| 10 | GATGAGTT[G/C]GATGATGAGTTGGAACAATTATAAGTTCA |
| | ATCTACTACGCCATACTTTAC (SEQ ID NO: 19/20) |
| SNP_ | ACTTATTTGTACCAGATGCTAGTTCATCATACGATCTCGATC |
| 11 | AACAGCTC[A/G]AAACTGTCCCCACTTCATCTGACGGCAAT |
| | ATCATGGTTTCTTGGAATCCT (SEQ ID NO: 21/22) |
| SNP_ | ACTCTTTTCAAAATGGCAATTCCAAAACTTGAACTTTAACTT |
| 12 | TTGTTACA[C/T]GCTTAATCACGTCGATAATCATGCTTAGC |
| | ACCACTGCCACTTTCTAAATC (SEQ ID NO: 23/24) |
| SNP_ | CCTGAACATTATGCAAAATGTTTAGCCACACTTTCACGCTCA |
| 13 | TCTTCACT[T/A]GGAATTCGATTGTGTTTTCGTCAGTACAG |
| | TAAAAATAAATTCAAGCTTTT (SEQ ID NO: 25/26) |
| SNP_ | CGTGAAAGTGTGGCTAAACATTTTGCATAATGTTCAGGAGTT |
| 14 | AAAACCAC[A/G]GCAATGTCATCTATACATTCTGCGCTGAT |
| | CACTTGTGAGAAGGGCGTGAA (SEQ ID NO: 27/28) |
| SNP_ | TAAGCTTCATGAAGCTATATATATCACAGTAAAACAGGTTAT |
| 15 | TTTTATGG[G/A]CATGGATTACTTTTTAAATTTGTAAGTTG |
| | GTTTTGTCTCCCTTTTGGTTA (SEQ ID NO: 29/30) |
| SNP_ | ACGTCGGCGGGAGCTTTCTCGGTTTGATACACGCCTAAATAG |
| 16 | CCGGTTGG[A/G]TCGACTCTCGCGTAGATCGGACCGTTGCC |
| | TTGGATTATTTGAGTTTTGGC (SEQ ID NO: 31/32) |
| SNP_ | TATAGTGTGGCAAAAGGTGCATTGCATAGAGCATTTGATGAG |
| 17 | ATAGTAGT[T/A]GTTGAAAGAAATTGTGGTCGAGAAGAGCA |
| | GAGAGATCCAATCAATATTAA (SEQ ID NO: 33/34) |
| SNP_ | TATTATAATTATAAGCAGTGATGTCACATTCATTAATTTGTG |
| 18 | CACCCTCA[A/T]TATTATCCCTCGATGAAAAGTCAATTATT |
| | TCGTTAGGAATATCCGTAGAC (SEQ ID NO: 35/36) |
| SNP_ | TGTTGCATTCCCCATTACCCAATATCACTGTCACAACTATGC |
| 19 | TCCCCAAC[A/T]CCTTGCTACACTGCAATAACATCACATAC |
| | AACTTCACTTCCCCGAACAAC (SEQ ID NO: 37/38) |
| SNP_ | CTTCCCCTCGGTAAATATTCTGTTACTATCGACAAATGTGGA |
| 20 | GACTTTGT[G/A]ACTGCACCCATAAATAGTACAATATTTGG |
| | ATGGCGAATACGTTTCATTAT (SEQ ID NO: 39/40) |
| SNP_ | TACTGCCCAGTCACTGCTTGTGGGGATGGATTCTTCGTCTTC |
| 21 | AAGCGATG[C/T]TCGAATTCTCTCAAGATCTTTCTCTGAAG |
| | CTTCTTTGAAATTAATGTCCT (SEQ ID NO: 41/42) |
| SNP_ | GATACCCAAAACCCTGATATGATCGACTATCTCAACCAAGAA |
| 22 | AATGATTA[C/T]ACTGAATCATTTATGAAAGATACTGAAAA |
| | ATTGCAGCGAAAATTAGTGGA (SEQ ID NO: 43/44) |
| SNP_ | TTATGGTCGAAAGAAGACCTATTGAACTTTGTCATAGCACCT |
| 23 | CCAGTGGG[T/C]ATCTTCCCACACAGCTTATTATAGCCGAC |
| | GTCAAAATACACCAGTTTGAT (SEQ ID NO: 45/46) |
| SNP_ | AAATTGGCTATGGAGAAGATGAAGATTGATTTGGCACAGAAA |
| 24 | GATAAGAT[C/G]CTGTCTGCATTGCTGAGAAAATCAAAGGC |
| | TGATAATGAAGAAAAGCATAT (SEQ ID NO: 47/48) |
| SNP_ | CATTCTGTCATGGTTATCAGTCACATCTAATGATGCTTTAAT |
| 25 | ACTTTCCG[G/A]ATTCAGAAATGACAAATGTGCTCCACCAA |
| | ATGCTTCTACACATGGTTTAC (SEQ ID NO: 49/50) |

### Example 9—Validation of QTL Markers

To validate that the identified markers are useful for predicting pyruvate level we genotyped a panel of lines with a range of pyruvate values. Five bulbs from each line were genotyped, and a pyruvate concentration was assigned based on the average for bulbs from that line.

Pyruvate measurements were made on bulbs after being stored for four months. A 5-10 mm thick slice was taken from the equator of an onion bulb (25-50 g). The slice was quartered, mixed with deionized water (1:10 dilution), and homogenized with an immersion blender until chunks disappeared (about 45 sec). One (1) mL of onion juice was centrifuged for 5 minutes (16,000×g at room temperature). Supernatant was used for pyruvate measurement, which was conducted according to Anthon and Barrett method (2003) with some modifications. Six (6) mL of onion juice was mixed with 50 mL of 0.025% dinitrophenylhydrazine (DNPH) reagent in a 96-well microplate. The mixture was incubated for 15 min at 37° C., and then mixed with 50 mL of 1.5N sodium hydroxide (NaOH). The mixture was cooled to room temperature before reading the absorbance at 515 nm. Pyruvate analysis was conducted in triplicate for each sample. A calibration curve was prepared using pyruvate standard solutions at 0.4, 0.8, and 1.2 mM. Results are reported in μmol of pyruvate per gram of fresh tissue (μmol/g).

The results of this validation confirmed that the reduced pungency haplotypes (“B” allele calls) were enriched in the reduced pungency lines, and the lines with lower average pyruvate values and less variation in pyruvate level had a lower occurrence of high pyruvate (“A”) alleles (Table 3). For all reduced pungency material, the Soluble Solids Content (SSC) as measured by Brix was above 7% and was not correlated with pyruvate level (R²=0.2 with a negative slope).

The linkage groups described herein above were mapped to known markers to determine which chromosome number corresponds to each of the linkage groups. It was accordingly found that linkage group 3 corresponds to chromosome 2 of Allium cepa, linkage group 4 corresponds to chromosome 1 of Allium cepa, and linkage group 6 corresponds to chromosome 7 of Allium cepa.

TABLE 9
Results of validation on reduced pungency plant material. Genotypes are coded in A/H/B format, where
A is the high pungency allele, H is heterozygous, and B is the reduced pungency allele. The
flanking SNP markers are underlined, whereas the peak markers are indicated in bold typeface (see
also Table 7 as provided herein above). The corresponding base call for the reduced pungency allele
is indicated in the row below the SNP marker names. Entries are ordered based on mean pyruvate
levels first, and then standard deviation of pyruvate levels when the mean level is similar.

| | | | | | | | |
|---|---|---|---|---|---|---|---|
| | Pyruvate | | Pyruvate | | | | |
| | mean | Pyruvate min | median | Pyruvate max | Pyruvate SD | | Number of |
| Line name | (μmol/g) | (μmol/g) | (μmol/g) | (μmol/g) | (μmol/g) | Brix mean | bulbs |

Base call for reduced pungency allele (B)

| | | | | | | | |
|---|---|---|---|---|---|---|---|
| Line 1 | 3.89 | 0.15 | 3.7 | 10.95 | 1.75 | 7.24 | 494 |
| Line 2 | 2.94 | 0.64 | 2.78 | 11.4 | 1.18 | 7.05 | 1213 |
| Line 3 | 2.88 | 0.88 | 2.73 | 6.67 | 1.18 | 7.88 | 185 |
| Line 4 | 1.97 | 0.33 | 1.84 | 9.7 | 0.82 | 7.83 | 950 |
| Line 5 | 2 | 0.69 | 1.9 | 4.4 | 0.65 | 7.95 | 148 |
| Line 6 | 1.48 | 0.35 | 1.3 | 4.35 | 0.69 | 7.4 | 405 |
| Line 7 | 1.47 | 0.46 | 1.38 | 6.24 | 0.57 | 7.69 | 489 |

Line name

| Base call | Linkage group 3 | | | | | | | | |
|---|---|---|---|---|---|---|---|---|---|
| for reduced | SNP_11 | SNP_12 | SNP_13 | SNP_14 | SNP_01 | SNP_02 | SNP_{—}03 | SNP_04 | SNP_15 |
| pungency allele (B) | A | C | T | A | T | A | C | T | G |
| Line 1 | H | H | B | B | A | H | B | B | B |
| | B | B | B | B | A | B | B | B | A |
| | H | H | B | B | A | B | B | H | A |
| | H | H | B | B | A | H | B | H | H |
| | B | B | B | B | A | H | B | H | B |
| Line 2 | H | . | B | B | H | H | H | H | B |
| | B | B | B | B | A | B | B | B | B |
| | B | B | B | B | A | B | B | B | B |
| | A | A | B | B | B | B | B | B | B |
| | H | H | A | H | A | A | A | A | B |
| Line 3 | H | B | B | B | B | B | B | B | B |
| | A | B | B | B | H | B | B | B | B |
| | B | B | B | B | H | B | B | B | B |
| | H | B | B | B | H | B | B | B | B |
| | A | B | B | B | H | B | B | B | B |
| Line 4 | B | B | B | B | B | B | B | B | B |
| | A | B | B | B | B | B | B | B | B |
| | B | B | B | B | B | B | B | B | B |
| | B | B | B | B | B | B | B | B | B |
| | B | B | B | B | B | B | B | B | B |
| Line 5 | B | B | B | B | B | B | B | B | B |
| | B | B | B | B | B | B | B | B | B |
| | B | B | B | B | B | B | B | B | B |
| | B | B | B | B | B | B | B | B | B |
| | B | B | B | B | B | B | B | B | B |
| Line 6 | B | B | B | B | B | B | B | B | B |
| | B | B | B | B | B | B | B | B | B |
| | B | B | B | B | B | B | B | B | B |
| | B | B | B | B | B | B | B | B | B |
| | B | B | B | B | B | B | B | B | B |
| Line 7 | B | B | B | B | B | B | B | B | B |
| | B | B | B | B | B | B | B | B | B |
| | B | B | B | B | B | B | B | B | B |
| | B | B | B | B | B | B | B | B | B |
| | B | B | B | B | B | B | B | B | B |

Line name

| Base call | Linkage group 4 | | | | | | | | | |
|---|---|---|---|---|---|---|---|---|---|---|
| for reduced | SNP_16 | SNP_17 | SNP_08 | SNP_06 | SNP_{—}07 | SNP_05 | SNP_18 | SNP_19 | SNP_20 | SNP_21 |
| pungency allele (B) | A | A | T | A | C | C | T | T | G | C |
| Line 1 | A | A | A | B | A | A | B | A | A | B |
| | A | A | A | B | A | A | B | A | A | B |
| | A | A | A | B | A | A | B | A | A | B |
| | A | A | A | B | A | A | B | A | A | B |
| | A | A | A | B | A | A | B | A | A | B |
| Line 2 | A | H | H | H | B | H | B | H | B | H |
| | A | A | H | B | A | A | B | A | A | B |
| | A | A | H | B | A | A | B | A | A | B |
| | A | B | B | B | B | B | H | B | H | B |
| | A | B | B | B | B | B | A | B | H | B |
| Line 3 | B | B | B | B | B | B | B | B | B | B |
| | B | H | B | B | B | B | B | B | B | B |
| | B | B | B | B | B | B | B | B | B | B |
| | B | H | B | B | B | B | B | B | B | B |
| | B | B | B | B | B | B | B | B | B | B |
| Line 4 | B | H | B | B | B | B | B | B | B | B |
| | B | B | B | B | B | B | B | B | B | B |
| | B | H | B | B | B | B | B | B | B | B |
| | B | H | B | B | B | B | B | B | B | B |
| | B | B | B | . | B | B | B | B | B | B |
| Line 5 | B | B | B | B | B | B | B | B | B | B |
| | B | B | B | B | B | B | B | B | B | B |
| | B | B | B | B | B | B | B | B | B | B |
| | B | B | B | B | B | B | B | B | B | B |
| | B | B | B | B | B | B | B | B | B | B |
| Line 6 | B | B | B | B | B | B | B | B | B | B |
| | B | B | B | B | B | B | B | B | B | B |
| | B | B | B | B | B | B | B | B | B | B |
| | B | B | B | B | B | B | B | B | B | B |
| | B | B | B | B | B | B | B | B | B | B |
| Line 7 | B | B | B | B | B | B | B | B | B | B |
| | B | B | B | B | B | B | B | B | B | B |
| | B | B | B | B | B | B | B | B | B | B |
| | B | B | B | B | B | B | B | B | B | B |
| | B | B | B | B | B | B | B | B | B | B |

| Line name | | | | | | |
|---|---|---|---|---|---|---|
| Base call | Linkage group 6 | | | | | |
| for reduced | SNP_22 | SNP_23 | SNP_24 | SNP_25 | SNP_09 | SNP_{—}10 |
| pungency allele (B) | T | T | C | G | T | G |
| Line 1 | B | B | B | A | A | B |
| | B | B | B | A | A | B |
| | B | B | B | A | A | B |
| | B | B | B | A | A | B |
| | B | B | B | A | A | B |
| Line 2 | A | B | A | H | H | H |
| | A | B | A | A | A | B |
| | A | B | A | A | A | B |
| | A | B | A | B | B | A |
| | A | B | A | B | B | H |
| Line 3 | A | B | B | B | B | H |
| | A | B | B | B | B | B |
| | B | B | B | B | B | H |
| | H | B | B | B | B | B |
| | H | B | B | B | B | B |
| Line 4 | B | B | B | B | B | H |
| | H | B | B | B | B | B |
| | H | B | B | B | B | H |
| | H | B | B | B | B | H |
| | B | B | B | B | B | B |
| Line 5 | B | B | B | B | B | B |
| | B | B | B | B | B | B |
| | B | B | B | B | B | B |
| | B | B | B | B | B | B |
| | B | B | B | B | B | B |
| Line 6 | B | B | B | B | B | B |
| | H | B | B | B | B | B |
| | B | B | B | B | B | B |
| | B | B | B | B | B | B |
| | B | B | B | B | B | B |
| Line 7 | B | B | B | B | B | B |
| | B | B | B | B | B | B |
| | B | B | B | B | B | B |
| | B | B | B | B | B | B |
| | B | B | B | B | B | B |

1. An Allium cepa plant, or plant part or seed thereof, comprising in its genome one or more of QTL1 on chromosome 1, QTL2 on chromosome 2 and/or QTL7 on chromosome 7, wherein said QTL1, QTL2 and QTL7 confer a reduced pyruvate level,
wherein QTL1 is comprised in an introgression fragment comprising a haplotype of one or more of the following SNP markers:
SNP_16 comprising an Adenine at nucleotide 51 of SEQ ID NO: 31 or at nucleotide 51 of a sequence comprising at least 97% identity to SEQ ID NO: 31;
SNP_17 comprising an Adenine at nucleotide 51 of SEQ ID NO: 33 or at nucleotide 51 of a sequence comprising at least 97% identity to SEQ ID NO: 33;
SNP_05 comprising a Cytosine at nucleotide 51 of SEQ ID NO: 9 or at nucleotide 51 of a sequence comprising at least 97% identity to SEQ ID NO: 9;
SNP_06 comprising an Adenine at nucleotide 51 of SEQ ID NO: 11 or at nucleotide 51 of a sequence comprising at least 97% identity to SEQ ID NO: 11;
SNP_07 comprising a Cytosine at nucleotide 51 of SEQ ID NO: 13 or at nucleotide 51 of a sequence comprising at least 97% identity to SEQ ID NO: 13;
SNP_08 comprising a Thymine at nucleotide 51 of SEQ ID NO: 15 or at nucleotide 51 of a sequence comprising at least 97% identity to SEQ ID NO: 15;
SNP_18 comprising a Thymine at nucleotide 51 of SEQ ID NO: 35 or at nucleotide 51 of a sequence comprising at least 97% identity to SEQ ID NO: 35;
SNP_19 comprising a Thymine at nucleotide 51 of SEQ ID NO: 37 or at nucleotide 51 of a sequence comprising at least 97% identity to SEQ ID NO: 37;
SNP_20 comprising a Guanine at nucleotide 51 of SEQ ID NO: 39 or at nucleotide 51 of a sequence comprising at least 97% identity to SEQ ID NO: 39; and/or
SNP_21 comprising a Cytosine at nucleotide 51 of SEQ ID NO: 41 or at nucleotide 51 of a sequence comprising at least 97% identity to SEQ ID NO: 41,
wherein QTL2 is comprised in an introgression fragment comprising a haplotype of one or more of the following SNP markers:
SNP_11 comprising an Adenine at nucleotide 51 of SEQ ID NO: 21 or at nucleotide 51 of a sequence comprising at least 97% identity to SEQ ID NO: 21;
SNP_12 comprising a Cytosine at nucleotide 51 of SEQ ID NO: 23 or at nucleotide 51 of a sequence comprising at least 97% identity to SEQ ID NO: 23;
SNP_13 comprising a Thymine at nucleotide 51 of SEQ ID NO: 25 or at nucleotide 51 of a sequence comprising at least 97% identity to SEQ ID NO: 25;
SNP_14 comprising an Adenine at nucleotide 51 of SEQ ID NO: 27 or at nucleotide 51 of a sequence comprising at least 97% identity to SEQ ID NO: 27;
SNP_01 comprising a Thymine at nucleotide 51 of SEQ ID NO: 1 or at nucleotide 51 of a sequence comprising at least 97% identity to SEQ ID NO: 1;
SNP_02 comprising an Adenine at nucleotide 51 of SEQ ID NO: 3 or at nucleotide 51 of a sequence comprising at least 97% identity to SEQ ID NO: 3;
SNP_03 comprising a Cytosine at nucleotide 51 of SEQ ID NO: 5 or at nucleotide 51 of a sequence comprising at least 97% identity to SEQ ID NO: 5;
SNP_04 comprising a Thymine at nucleotide 51 of SEQ ID NO: 7 or at nucleotide 51 of a sequence comprising at least 97% identity to SEQ ID NO: 7; and/or
SNP_15 comprising a Guanine at nucleotide 51 of SEQ ID NO: 29 or at nucleotide 51 of a sequence comprising at least 97% identity to SEQ ID NO: 29, and/or
wherein QTL7 is comprised in an introgression fragment comprising a haplotype of one or more of the following SNP markers:
SNP_22 comprising a Thymine at nucleotide 51 of SEQ ID NO: 43 or at nucleotide 51 of a sequence comprising at least 97% identity to SEQ ID NO: 43;
SNP_23 comprising a Thymine at nucleotide 51 of SEQ ID NO: 45 or at nucleotide 51 of a sequence comprising at least 97% identity to SEQ ID NO: 45;
SNP_24 comprising a Cytosine at nucleotide 51 of SEQ ID NO: 47 or at nucleotide 51 of a sequence comprising at least 97% identity to SEQ ID NO: 47;
SNP_25 comprising a Guanine at nucleotide 51 of SEQ ID NO: 49 or at nucleotide 51 of a sequence comprising at least 97% identity to SEQ ID NO: 49;
SNP_09 comprising a Thymine at nucleotide 51 of SEQ ID NO: 17 or at nucleotide 51 of a sequence comprising at least 97% identity to SEQ ID NO: 17; and/or
SNP_10 comprising a Guanine at nucleotide 51 of SEQ ID NO: 19 or at nucleotide 51 of a sequence comprising at least 97% identity to SEQ ID NO: 19.

2. The plant of claim 1, comprising two or more of QTL1 on chromosome 1, QTL2 on chromosome 2 and/or QTL7 on chromosome 7.

3. The plant of claim 1, comprising QTL1 on chromosome 1, QTL2 on chromosome 2 and QTL7 on chromosome 7.

4. The plant of claim 1, wherein QTL1 is comprised in an introgression fragment comprising a haplotype of two or more of the following SNP markers: SNP_16, SNP_17, SNP_05, SNP_06, SNP_07, SNP_08, SNP_18, SNP_19, SNP_20 and/or SNP_21.

5. The plant of claim 1, wherein QTL1 is comprised in an introgression fragment comprising a haplotype of 3 or more of the following SNP markers: SNP_16, SNP_17, SNP_05, SNP_06, SNP_07, SNP_08, SNP_18, SNP_19, SNP_20 and/or SNP_21.

6. The plant of claim 1, wherein QTL2 is comprised in an introgression fragment comprising a haplotype of two or more of the following SNP markers: SNP_11, SNP_12, SNP_13, SNP_14, SNP_01, SNP_02, SNP_03, SNP_04 and/or SNP_15.

7. The plant of claim 1, wherein QTL2 is comprised in an introgression fragment comprising a haplotype of 3 or more of the following SNP markers: SNP_11, SNP_12, SNP_13, SNP_14, SNP_01, SNP_02, SNP_03, SNP_04 and/or SNP_15.

8. The plant of claim 1, wherein QTL7 is comprised in an introgression fragment comprising a haplotype of two or more of the following SNP markers: SNP_22, SNP_23, SNP_24, SNP_25, SNP_09 and/or SNP_10.

9. The plant of claim 1, wherein QTL7 is comprised in an introgression fragment comprising a haplotype of 3 or more of the following SNP markers: SNP_22, SNP_23, SNP_24, SNP_25, SNP_09 and/or SNP_10.

10. The plant of claim 1, wherein the plant is a single cross F1 hybrid or an inbred line.

11. A method of producing an Allium cepa plant comprising in its genome one or more of QTL1 on chromosome 1, QTL2 on chromosome 2 and/or QTL7 on chromosome 7, wherein said QTL1, QTL2 and QTL7 confer a reduced pyruvate level, said method comprising:
a) crossing a first onion plant comprising in its genome one or more of QTL1, QTL2 and/or QTL7 with a second onion plant, and b) collecting seeds from said cross,
wherein QTL1 is comprised in an introgression fragment comprising a haplotype of one or more of the following SNP markers:
SNP_16 comprising an Adenine at nucleotide 51 of SEQ ID NO: 31 or at nucleotide 51 of a sequence comprising at least 97% identity to SEQ ID NO: 31;
SNP_17 comprising an Adenine at nucleotide 51 of SEQ ID NO: 33 or at nucleotide 51 of a sequence comprising at least 97% identity to SEQ ID NO: 33;
SNP_05 comprising a Cytosine at nucleotide 51 of SEQ ID NO: 9 or at nucleotide 51 of a sequence comprising at least 97% identity to SEQ ID NO: 9;
SNP_06 comprising an Adenine at nucleotide 51 of SEQ ID NO: 11 or at nucleotide 51 of a sequence comprising at least 97% identity to SEQ ID NO: 11;
SNP_07 comprising a Cytosine at nucleotide 51 of SEQ ID NO: 13 or at nucleotide 51 of a sequence comprising at least 97% identity to SEQ ID NO: 13;
SNP_08 comprising a Thymine at nucleotide 51 of SEQ ID NO: 15 or at nucleotide 51 of a sequence comprising at least 97% identity to SEQ ID NO: 15;
SNP_18 comprising a Thymine at nucleotide 51 of SEQ ID NO: 35 or at nucleotide 51 of a sequence comprising at least 97% identity to SEQ ID NO: 35;
SNP_19 comprising a Thymine at nucleotide 51 of SEQ ID NO: 37 or at nucleotide 51 of a sequence comprising at least 97% identity to SEQ ID NO: 37;
SNP_20 comprising a Guanine at nucleotide 51 of SEQ ID NO: 39 or at nucleotide 51 of a sequence comprising at least 97% identity to SEQ ID NO: 39; and/or
SNP_21 comprising a Cytosine at nucleotide 51 of SEQ ID NO: 41 or at nucleotide 51 of a sequence comprising at least 97% identity to SEQ ID NO: 41,
wherein QTL2 is comprised in an introgression fragment comprising a haplotype of one or more of the following SNP markers:
SNP_11 comprising an Adenine at nucleotide 51 of SEQ ID NO: 21 or at nucleotide 51 of a sequence comprising at least 97% identity to SEQ ID NO: 21;
SNP_12 comprising a Cytosine at nucleotide 51 of SEQ ID NO: 23 or at nucleotide 51 of a sequence comprising at least 97% identity to SEQ ID NO: 23;
SNP_13 comprising a Thymine at nucleotide 51 of SEQ ID NO: 25 or at nucleotide 51 of a sequence comprising at least 97% identity to SEQ ID NO: 25;
SNP_14 comprising an Adenine at nucleotide 51 of SEQ ID NO: 27 or at nucleotide 51 of a sequence comprising at least 97% identity to SEQ ID NO: 27;
SNP_01 comprising a Thymine at nucleotide 51 of SEQ ID NO: 1 or at nucleotide 51 of a sequence comprising at least 97% identity to SEQ ID NO: 1;
SNP_02 comprising an Adenine at nucleotide 51 of SEQ ID NO: 3 or at nucleotide 51 of a sequence comprising at least 97% identity to SEQ ID NO: 3;
SNP_03 comprising a Cytosine at nucleotide 51 of SEQ ID NO: 5 or at nucleotide 51 of a sequence comprising at least 97% identity to SEQ ID NO: 5;
SNP_04 comprising a Thymine at nucleotide 51 of SEQ ID NO: 7 or at nucleotide 51 of a sequence comprising at least 97% identity to SEQ ID NO: 7; and/or
SNP_15 comprising a Guanine at nucleotide 51 of SEQ ID NO: 29 or at nucleotide 51 of a sequence comprising at least 97% identity to SEQ ID NO: 29, and/or
wherein QTL7 is comprised in an introgression fragment comprising a haplotype of one or more of the following SNP markers:
SNP_22 comprising a Thymine at nucleotide 51 of SEQ ID NO: 43 or at nucleotide 51 of a sequence comprising at least 97% identity to SEQ ID NO: 43;
SNP_23 comprising a Thymine at nucleotide 51 of SEQ ID NO: 45 or at nucleotide 51 of a sequence comprising at least 97% identity to SEQ ID NO: 45;
SNP_24 comprising a Cytosine at nucleotide 51 of SEQ ID NO: 47 or at nucleotide 51 of a sequence comprising at least 97% identity to SEQ ID NO: 47;
SNP_25 comprising a Guanine at nucleotide 51 of SEQ ID NO: 49 or at nucleotide 51 of a sequence comprising at least 97% identity to SEQ ID NO: 49;
SNP_09 comprising a Thymine at nucleotide 51 of SEQ ID NO: 17 or at nucleotide 51 of a sequence comprising at least 97% identity to SEQ ID NO: 17; and/or
SNP_10 comprising a Guanine at nucleotide 51 of SEQ ID NO: 19 or at nucleotide 51 of a sequence comprising at least 97% identity to SEQ ID NO: 19.

12. The method of claim 11, wherein the first onion plant comprises two or more of QTL1 on chromosome 1, QTL2 on chromosome 2 and/or QTL7 on chromosome 7.

13. The method of claim 11, wherein the first onion plant comprises QTL1 on chromosome 1, QTL2 on chromosome 2 and QTL7 on chromosome 7.

14. The method of claim 11, wherein QTL1 is comprised in an introgression fragment comprising a haplotype of two or more of the following SNP markers: SNP_16, SNP_17, SNP_05, SNP_06, SNP_07, SNP_08, SNP_18, SNP_19, SNP_20 and/or SNP_21.

15. The method of claim 11, wherein QTL1 is comprised in an introgression fragment comprising a haplotype of 3 or more of the following SNP markers: SNP_16, SNP_17, SNP_05, SNP_06, SNP_07, SNP_08, SNP_18, SNP_19, SNP_20 and/or SNP_21.

16. The method of claim 11, wherein QTL2 is comprised in an introgression fragment comprising a haplotype of two or more of the following SNP markers: SNP_11, SNP_12, SNP_13, SNP_14, SNP_01, SNP_02, SNP_03, SNP_04 and/or SNP_15.

17. The method of claim 11, wherein QTL2 is comprised in an introgression fragment comprising a haplotype of 3 or more of the following SNP markers: SNP_11, SNP_12, SNP_13, SNP_14, SNP_01, SNP_02, SNP_03, SNP_04 and/or SNP_15.

18. The method of claim 11, wherein QTL7 is comprised in an introgression fragment comprising a haplotype of two or more of the following SNP markers: SNP_22, SNP_23, SNP_24, SNP_25, SNP_09 and/or SNP_10.

19. The method of claim 11, wherein QTL7 is comprised in an introgression fragment comprising a haplotype of 3 or more of the following SNP markers: SNP_22, SNP_23, SNP_24, SNP_25, SNP_09/or SNP_10.

20. The method of claim 11, wherein the produced Allium cepa plant is a single cross F1 hybrid or an inbred line.
